(19) United States
(12) Patent Application Publication
(10) Pub. No.: US 2024/0225660 A1
(43) Pub. Date: Jul. 11, 2024

(54) Systems and Methods for Forming and Deploying the Janjua Aneurysm Net plus Bridge (JAN-B) for Cerebral Aneurysm Occlusion

(71) Applicant: Robert A. Connor, Wyoming, MN (US)

(72) Inventor: Robert A. Connor, Wyoming, MN (US)

(73) Assignee: Aneuclose LLC, Ham Lake, MN (US)

(21) Appl. No.: 18/613,053

(22) Filed: Mar. 21, 2024

Related U.S. Application Data
(63) Continuation-in-part of application No. 18/519,055, filed on Nov. 26, 2023.
(63) Continuation-in-part of application No. 18/135,153, filed on Apr. 15, 2023.
Application No. 18/519,055 is a continuation-in-part of application No. 18/374,602, filed on Sep. 28, 2023, now Pat. No. 11,944,316.
Application No. 18/374,602 is a continuation-in-part of application No. 18/135,153, filed on Apr. 15, 2023.
Application No. 18/135,153 is a continuation-in-part of application No. 17/970,510, filed on Oct. 20, 2022.
Application No. 17/970,510 is a continuation-in-part of application No. 17/965,502, filed on Oct. 13, 2022, now abandoned.
Application No. 17/965,502 is a continuation-in-part of application No. 17/829,313, filed on May 31, 2022, now abandoned.
Application No. 18/374,602 is a continuation-in-part of application No. 18/135,153, filed on Apr. 15, 2023.
Application No. 18/135,153 is a continuation-in-part of application No. 17/970,510, filed on Oct. 20, 2022.
Application No. 17/970,510 is a continuation-in-part of application No. 17/965,502, filed on Oct. 13, 2022, now abandoned.
Application No. 17/965,502 is a continuation-in-part of application No. 17/829,313, filed on May 31, 2022, now abandoned.
Application No. 18/135,153 is a continuation-in-part of application No. 17/970,510, filed on Oct. 20, 2022.
Application No. 17/970,510 is a continuation-in-part of application No. 17/965,502, filed on Oct. 13, 2022, now abandoned.
Application No. 17/965,502 is a continuation-in-part of application No. 17/829,313, filed on May 31, 2022, now abandoned.
Application No. 17/970,510 is a continuation-in-part of application No. 17/965,502, filed on Oct. 13, 2022, now abandoned.
Application No. 17/965,502 is a continuation-in-part of application No. 17/829,313, filed on May 31, 2022, now abandoned.
Application No. 17/829,313 is a continuation-in-part of application No. 17/476,845, filed on Sep. 16, 2021, now Pat. No. 11,583,289.
Application No. 17/965,502 is a continuation-in-part of application No. 17/829,313, filed on May 31, 2022, now abandoned.
Application No. 17/829,313 is a continuation-in-part of application No. 17/476,845, filed on Sep. 16, 2021, now Pat. No. 11,583,289.
Application No. 17/829,313 is a continuation-in-part of application No. 17/485,390, filed on Sep. 25, 2021, now Pat. No. 11,471,164.
Application No. 17/485,390 is a continuation-in-part of application No. 17/476,845, filed on Sep. 16, 2021, now Pat. No. 11,583,289.
Application No. 17/476,845 is a continuation-in-part of application No. 17/472,674, filed on Sep. 12, 2021, now abandoned.
Application No. 17/472,674 is a continuation-in-part of application No. 17/467,680, filed on Sep. 7, 2021, now abandoned.
Application No. 17/467,680 is a continuation-in-part of application No. 17/466,497, filed on Sep. 3, 2021, now Pat. No. 11,357,511.
Application No. 17/466,497 is a continuation-in-part of application No. 17/353,652, filed on Jun. 21, 2021, now abandoned.
Application No. 17/353,652 is a continuation-in-part of application No. 17/220,002, filed on Apr. 1, 2021, now Pat. No. 11,464,518.
Application No. 17/220,002 is a continuation-in-part of application No. 17/214,827, filed on Mar. 27, 2021, now Pat. No. 11,484,322.
Application No. 17/214,827 is a continuation-in-part of application No. 17/211,446, filed on Mar. 24, 2021, now abandoned.
Application No. 17/220,002 is a continuation-in-part of application No. 17/214,827, filed on Mar. 27, 2021, now Pat. No. 11,484,322.
Application No. 17/214,827 is a continuation-in-part of application No. 17/211,446, filed on Mar. 24, 2021, now abandoned.
Application No. 17/211,446 is a continuation-in-part of application No. 16/693,267, filed on Nov. 23, 2019, now abandoned.
Application No. 16/693,267 is a continuation-in-part of application No. 16/660,929, filed on Oct. 23, 2019, now Pat. No. 11,471,163.
Application No. 16/693,267 is a continuation-in-part of application No. 16/660,929, filed on Oct. 23, 2019, now Pat. No. 11,471,163.
Application No. 16/660,929 is a continuation-in-part of application No. 16/541,241, filed on Aug. 15, 2019, now abandoned.
Application No. 16/541,241 is a continuation-in-part of application No. 15/865,822, filed on Jan. 9, 2018, now Pat. No. 10,716,573.
Application No. 15/865,822 is a continuation-in-part of application No. 15/861,482, filed on Jan. 3, 2018, now abandoned.
Application No. 15/861,482 is a continuation-in-part of application No. 16/541,241, filed on Aug. 15, 2019, now abandoned.
Application No. 16/541,241 is a continuation-in-part of application No. 15/865,822, filed on Jan. 9, 2018, now Pat. No. 10,716,573.
Application No. 15/865,822 is a continuation-in-part of application No. 15/861,482, filed on Jan. 3, 2018, now abandoned.
Application No. 15/861,482 is a continuation-in-part of application No. 15/865,822, filed on Jan. 9, 2018, now Pat. No. 10,716,573.
Application No. 15/865,822 is a continuation-in-part of application No. 15/081,909, filed on Mar. 27, 2016, now abandoned.
Application No. 15/081,909 is a continuation-in-part of application No. 14/526,600, filed on Oct. 29, 2014, now abandoned.
Application No. 14/526,600 is a continuation-in-part of application No. 15/080,915, filed on Mar. 25, 2016, now Pat. No. 10,028,747.
Application No. 15/080,915 is a continuation-in-part of application No. 14/526,600, filed on Oct. 29, 2014, now abandoned.
Application No. 15/081,909 is a continuation-in-part of application No. 14/526,600, filed on Oct. 29, 2014, now abandoned.
Application No. 15/080,915 is a continuation-in-part of application No. 14/526,600, filed on Oct. 29, 2014, now abandoned.
Application No. 14/526,600 is a continuation-in-part of application No. 12/989,048, filed on Oct. 21, 2010, now Pat. No. 8,974,487.
(60) Provisional application No. 63/119,774, filed on Dec. 1, 2020.
(60) Provisional application No. 62/794,609, filed on Jan. 19, 2019.
(60) Provisional application No. 62/794,607, filed on Jan. 19, 2019.
(60) Provisional application No. 62/794,609, filed on Jan. 19, 2019.
(60) Provisional application No. 62/794,607, filed on Jan. 19, 2019.
(60) Provisional application No. 62/794,609, filed on Jan. 19, 2019.
(60) Provisional application No. 62/794,607, filed on Jan. 19, 2019.
(60) Provisional application No. 62/720,173, filed on Aug. 21, 2018.
(60) Provisional application No. 62/589,754, filed on Nov. 22, 2017.
(60) Provisional application No. 62/472,519, filed on Mar. 16, 2017.
(60) Provisional application No. 62/589,754, filed on Nov. 22, 2017.
(60) Provisional application No. 62/472,519, filed on Mar. 16, 2017.
(60) Provisional application No. 62/444,860, filed on Jan. 11, 2017.
(60) Provisional application No. 61/897,245, filed on Oct. 30, 2013.
(60) Provisional application No. 61/126,047, filed on May 1, 2008.
(60) Provisional application No. 61/126,027, filed on May 1, 2008.

(51) Int. Cl. A61B 17/12; A61B 17/00

(52) U.S. Cl. CPC A61B 17/12172; A61B 17/12113; A61B 17/12177; A61B 2017/00292; A61B 2017/12054

(57) ABSTRACT

Disclosed herein are systems and methods for forming and deploying an intrasaccular occlusion system which includes: a proximal neck bridge which expands within an aneurysm sac to span the aneurysm neck; a distal net or mesh which is expanded within the aneurysm sac between the proximal neck bridge and the aneurysm dome; and embolic members which are inserted into the net or mesh, wherein insertion of the embolic members expands the net or mesh.

### CROSS-REFERENCE TO RELATED APPLICATIONS

This application is a continuation-in-part of U.S. patent application Ser. No. 18/519,055 filed on Nov. 26, 2023 and a continuation-in-part of U.S. patent application Ser. No. 18/135,153 filed on Apr. 15, 2023.

U.S. patent application Ser. No. 18/519,055 was a continuation-in-part of U.S. patent application Ser. No. 18/374,602 filed on Sep. 28, 2023, a continuation-in-part of U.S. patent application Ser. No. 18/135,153 filed on Apr. 15, 2023, a continuation-in-part of U.S. patent application Ser. No. 17/970,510 filed on Oct. 20, 2022, a continuation-in-part of U.S. patent application Ser. No. 17/965,502 filed on Oct. 13, 2022, and a continuation-in-part of U.S. patent application Ser. No. 17/829,313 filed on May 31, 2022.

U.S. patent application Ser. No. 18/374,602 was a continuation-in-part of U.S. patent application Ser. No. 18/135,153 filed on Apr. 15, 2023, a continuation-in-part of U.S. patent application Ser. No. 17/970,510 filed on Oct. 20, 2022, a continuation-in-part of U.S. patent application Ser. No. 17/965,502 filed on Oct. 13, 2022, and a continuation-in-part of U.S. patent application Ser. No. 17/829,313 filed on May 31, 2022.

U.S. patent application Ser. No. 18/135,153 was a continuation-in-part of U.S. patent application Ser. No. 17/970,510 filed on Oct. 20, 2022, a continuation-in-part of U.S. patent application Ser. No. 17/965,502 filed on Oct. 13, 2022, and a continuation-in-part of U.S. patent application Ser. No. 17/829,313 filed on May 31, 2022. U.S. patent application Ser. No. 17/970,510 was a continuation-in-part of U.S. patent application Ser. No. 17/965,502 filed on Oct. 13, 2022, a continuation-in-part of U.S. patent application Ser. No. 17/829,313 filed on May 31, 2022, and a continuation-in-part of U.S. patent application Ser. No. 17/476,845 filed on Sep. 16, 2021.

U.S. patent application Ser. No. 17/829,313 was a continuation-in-part of U.S. patent application Ser. No. 17/485,390 filed on Sep. 25, 2021, was a continuation-in-part of U.S. patent application Ser. No. 17/476,845 filed on Sep. 16, 2021, was a continuation-in-part of U.S. patent application Ser. No. 17/472,674 filed on Sep. 12, 2021, was a continuation-in-part of U.S. patent application Ser. No. 17/467,680 filed on Sep. 7, 2021, was a continuation-in-part of U.S. patent application Ser. No. 17/466,497 filed on Sep. 3, 2021, was a continuation-in-part of U.S. patent application Ser. No. 17/353,652 filed on Jun. 21, 2021, was a continuation-in-part of U.S. patent application Ser. No. 17/220,002 filed on Apr. 1, 2021, was a continuation-in-part of U.S. patent application Ser. No. 17/214,827 filed on Mar. 27, 2021, was a continuation-in-part of U.S. patent application Ser. No. 17/211,446 filed on Mar. 24, 2021, was a continuation-in-part of U.S. patent application Ser. No. 16/693,267 filed on Nov. 23, 2019, and was a continuation-in-part of U.S. patent application Ser. No. 16/660,929 filed on Oct. 23, 2019.

U.S. patent application Ser. No. 17/220,002 was a continuation-in-part of U.S. patent application Ser. No. 17/214,827 filed on Mar. 27, 2021. U.S. patent application Ser. No. 17/220,002 was a continuation-in-part of U.S. patent application Ser. No. 17/211,446 filed on Mar. 24, 2021. U.S. patent application Ser. No. 17/220,002 claimed the priority benefit of U.S. provisional patent application 63/119,774 filed on Dec. 1, 2020. U.S. patent application Ser. No. 17/220,002 was a continuation-in-part of U.S. patent application Ser. No. 16/693,267 filed on Nov. 23, 2019. U.S. patent application Ser. No. 17/220,002 was a continuation-in-part of U.S. patent application Ser. No. 16/660,929 filed on Oct. 23, 2019.

U.S. patent application Ser. No. 16/693,267 was a continuation-in-part of U.S. patent application Ser. No. 16/660,929 filed on Oct. 23, 2019. U.S. patent application Ser. No. 16/693,267 claimed the priority benefit of U.S. provisional patent application 62/794,609 filed on Jan. 19, 2019. U.S. patent application Ser. No. 16/693,267 claimed the priority benefit of U.S. provisional patent application 62/794,607 filed on Jan. 19, 2019. U.S. patent application Ser. No. 16/693,267 was a continuation-in-part of U.S. patent application Ser. No. 16/541,241 filed on Aug. 15, 2019. U.S. patent application Ser. No. 16/693,267 was a continuation-in-part of U.S. patent application Ser. No. 15/865,822 filed on Jan. 9, 2018 which issued as U.S. Pat. No. 10,716,573 on Jul. 21, 2020. U.S. patent application Ser. No. 16/693,267 was a continuation-in-part of U.S. patent application Ser. No. 15/861,482 filed on Jan. 3, 2018.

U.S. patent application Ser. No. 16/660,929 claimed the priority benefit of U.S. provisional patent application 62/794,609 filed on Jan. 19, 2019. U.S. patent application Ser. No. 16/660,929 claimed the priority benefit of U.S. provisional patent application 62/794,607 filed on Jan. 19, 2019. U.S. patent application Ser. No. 16/660,929 was a continuation-in-part of U.S. patent application Ser. No. 16/541,241 filed on Aug. 15, 2019. U.S. patent application Ser. No. 16/660,929 was a continuation-in-part of U.S. patent application Ser. No. 15/865,822 filed on Jan. 9, 2018 which issued as U.S. Pat. No. 10,716,573 on Jul. 21, 2020. U.S. patent application Ser. No. 16/660,929 was a continuation-in-part of U.S. patent application Ser. No. 15/861,482 filed on Jan. 3, 2018.

U.S. patent application Ser. No. 16/541,241 claimed the priority benefit of U.S. provisional patent application 62/794,609 filed on Jan. 19, 2019. U.S. patent application Ser. No. 16/541,241 claimed the priority benefit of U.S. provisional patent application 62/794,607 filed on Jan. 19, 2019. U.S. patent application Ser. No. 16/541,241 claimed the priority benefit of U.S. provisional patent application 62/720,173 filed on Aug. 21, 2018. U.S. patent application Ser. No. 16/541,241 was a continuation-in-part of U.S. patent application Ser. No. 15/865,822 filed on Jan. 9, 2018 which issued as U.S. Pat. No. 10,716,573 on Jul. 21, 2020

U.S. patent application Ser. No. 15/865,822 claimed the priority benefit of U.S. provisional patent application 62/589,754 filed on Nov. 22, 2017. U.S. patent application Ser. No. 15/865,822 claimed the priority benefit of U.S. provisional patent application 62/472,519 filed on Mar. 16, 2017. U.S. patent application Ser. No. 15/865,822 was a continuation-in-part of U.S. patent application Ser. No. 15/081,909 filed on Mar. 27, 2016. U.S. patent application Ser. No. 15/865,822 was a continuation-in-part of U.S. patent application Ser. No. 14/526,600 filed on Oct. 29, 2014.

U.S. patent application Ser. No. 15/861,482 claimed the priority benefit of U.S. provisional patent application 62/589,754 filed on Nov. 22, 2017. U.S. patent application Ser. No. 15/861,482 claimed the priority benefit of U.S. provisional patent application 62/472,519 filed on Mar. 16, 2017. U.S. patent application Ser. No. 15/861,482 claimed the priority benefit of U.S. provisional patent application 62/444,860 filed on Jan. 11, 2017. U.S. patent application Ser. No. 15/861,482 was a continuation-in-part of U.S. patent application Ser. No. 15/080,915 filed on Mar. 25, 2016 which issued as U.S. Pat. No. 10,028,747 on Jul. 24, 2018. U.S. patent application Ser. No. 15/861,482 was a continuation-in-part of U.S. patent application Ser. No. 14/526,600 filed on Oct. 29, 2014.

U.S. patent application Ser. No. 15/081,909 was a continuation-in-part of U.S. patent application Ser. No. 14/526,600 filed on Oct. 29, 2014. U.S. patent application Ser. No. 15/080,915 was a continuation-in-part of U.S. patent application Ser. No. 14/526,600 filed on Oct. 29, 2014. U.S. patent application Ser. No. 14/526,600 claimed the priority benefit of U.S. provisional patent application 61/897,245 filed on Oct. 30, 2013. U.S. patent application Ser. No. 14/526,600 was a continuation-in-part of U.S. patent application Ser. No. 12/989,048 filed on Oct. 21, 2010 which issued as U.S. Pat. No. 8,974,487 on Mar. 10, 2015. U.S. patent application Ser. No. 12/989,048 claimed the priority benefit of U.S. provisional patent application 61/126,047 filed on May 1, 2008. U.S. patent application Ser. No. 12/989,048 claimed the priority benefit of U.S. provisional patent application 61/126,027 filed on May 1, 2008.

The entire contents of these related applications are incorporated herein by reference.

### FEDERALLY SPONSORED RESEARCH

Not Applicable

### SEQUENCE LISTING OR PROGRAM

Not Applicable

### BACKGROUND

### Field of Invention

This invention relates to aneurysm occlusion devices and methods.

### INTRODUCTION

An aneurysm is an abnormal bulging of a blood vessel wall. The vessel from which the aneurysm protrudes is the parent vessel. Saccular aneurysms look like a sac protruding out from the parent vessel. Saccular aneurysms have a neck and can be prone to rupture. Fusiform aneurysms are a form of aneurysm in which a blood vessel is expanded circumferentially in all directions. Fusiform aneurysms generally do not have a neck and are less prone to rupturing than saccular aneurysms. As an aneurysm grows larger, its walls generally become thinner and weaker. This decrease in wall integrity, particularly for saccular aneurysms, increases the risk of the aneurysm rupturing and hemorrhaging blood into the surrounding tissue, with serious and potentially fatal health outcomes.

Cerebral aneurysms, also called brain aneurysms or intracranial aneurysms, are aneurysms that occur in the intercerebral arteries that supply blood to the brain. The majority of cerebral aneurysms form at the junction of arteries at the base of the brain that is known as the Circle of Willis where arteries come together and from which these arteries send branches to different areas of the brain. Although identification of intact aneurysms is increasing due to increased use of outpatient imaging such as outpatient MRI scanning, many cerebral aneurysms still remain undetected unless they rupture. If they do rupture, they often cause stroke, disability, and/or death. The prevalence of cerebral aneurysms is generally estimated to be in the range of 1%-5% of the general population or approximately 3-15 million people in the U.S. alone. Approximately 30,000 people per year suffer a ruptured cerebral aneurysm in the U.S. alone. Approximately one-third to one-half of people who suffer a ruptured cerebral aneurysm die within one month of the rupture. Sadly, even among those who survive, approximately one-half suffer significant and permanent deterioration of brain function. Better alternatives for cerebral aneurysm treatment are needed.

### REVIEW OF THE RELEVANT ART

U.S. Pat. No. 8,998,947 (Aboytes et al., Apr. 7, 2015, “Devices and Methods for the Treatment of Vascular Defects”) discloses an expandable implant that with a first configuration in which the first portion and the second portion are substantially linearly aligned and a second configuration in which the second portion at least partially overlaps the first portion. U.S. patent application 20200205841 (Aboytes et al., Jul. 2, 2020, “Devices, Systems, and Methods for the Treatment of Vascular Defects”) and U.S. patent application 20210378681 (Aboytes et al., Dec. 9, 2021, “Devices, Systems, and Methods for the Treatment of Vascular Defects”) disclose aneurysm occlusion devices with a first configuration in which a first portion and a second portion are substantially linearly aligned and a second configuration in which the second portion at least partially overlaps the first portion. U.S. patent application 20210244420 (Aboytes et al., Aug. 12, 2021, “Devices and Methods for the Treatment of Vascular Defects”) discloses aneurysm occlusion devices with a first configuration in which a first portion and a second portion are substantially linearly aligned and a second configuration in which the second portion at least partially overlaps the first portion.

U.S. patent application 20220031334 (Aguilar, Feb. 3, 2022, “Expandable Devices for Treating Body Lumens”) discloses an occlusive device comprising an expandable mesh including an outer mesh and an inner mesh disposed within the outer mesh. U.S. patent application 20230240686 (Ashby et al., Aug. 3, 2023, “Occlusive Devices with Spiral Struts for Treating Vascular Defects”) discloses an occlusive device with a plurality of spiral struts. U.S. Pat. No. 11,185,335 (Badruddin et al., Nov. 30, 2021, “System for and Method of Treating Aneurysms”) discloses an apparatus for treating an aneurysm with an occlusion element disposed on a wire, wherein the occlusion element includes a cover for covering a neck of an aneurysm and an inner anchoring member. U.S. patent application 20170079661 (Bardsley et al., Mar. 23, 2017, “Occlusive Devices”), U.S. Pat. No. 10,314,593 (Bardsley et al., Jun. 11, 2019, “Occlusive Devices”), and U.S. patent application 20190269411 (Bardsley et al., Sep. 5, 2019, “Occlusive Devices”) disclose an implant with a single- or dual-layer braided body with variable porosity.

U.S. patent application 20110208227 (Becking, Aug. 25, 2011, “Filamentary Devices for Treatment of Vascular Defects”) discloses braid-balls for aneurysm occlusion. U.S. patent application 20120316598 (Becking et al., Dec. 13, 2012, “Multiple Layer Filamentary Devices for Treatment of Vascular Defects”) and U.S. Pat. No. 9,585,669 (Becking et al., Mar. 17, 2017, “Multiple Layer Filamentary Devices for Treatment of Vascular Defects”) disclose braid balls for aneurysm occlusion. U.S. Pat. No. 9,039,726 (Becking, May 26, 2015, “Filamentary Devices for Treatment of Vascular Defects”) discloses braid-balls for aneurysm occlusion. U.S. patent application 20200367904 (Becking et al., Nov. 26, 2020, “Multiple Layer Filamentary Devices for Treatment of Vascular Defects”) and U.S. patent application 20220022886 (Becking et al., Jan. 27, 2022, “Multiple Layer Filamentary Devices for Treatment of Vascular Defects”) disclose braid-balls suitable for aneurysm occlusion.

U.S. patent application 20210228214 (Bowman et al., Jul. 29, 2021, “Devices for Vascular Occlusion”) discloses a method of using and delivering an occlusive device. U.S. patent application 20230157696 (Carrillo, May 25, 2023, “Aneurysm Treatment Device and Associated Systems and Methods of Use”) discloses an aneurysm treatment device with a tip portion, a body portion, and a base portion. U.S. patent application 20230165587 (Carrillo, Jun. 1, 2023, “Expandable Devices for Treating Body Lumens”) discloses an expandable cage with a plurality of mesh stents which receives embolic material therein. U.S. patent application 20220370078 (Chen et al., Nov. 24, 2022, “Vaso-Occlusive Devices”) discloses a vaso-occlusive structure made with a gold-platinum-tungsten alloy. U.S. patent application 20230190292 (Choubey et al., Jun. 22, 2023, “Occlusive Devices with Petal-Shaped Regions for Treating Vascular Defects”) discloses an occlusive device for treating an aneurysm with a mesh formed from a tubular braid, including a petal-shaped region formed from a flattened section of the tubular braid.

U.S. patent application 20120165919 (Cox et al., Jun. 28, 2012, “Methods and Devices for Treatment of Vascular Defects”) and U.S. patent application 20140052233 (Cox et al., Feb. 20, 2014, “Methods and Devices for Treatment of Vascular Defects”) disclose an expandable wire body support structure having a low profile radially constrained state, an expanded relaxed state with a substantially spherical or globular configuration having a smooth outer surface, and a porous permeable layer comprising a braided wire occlusive mesh. U.S. patent application 20120283768 (Cox et al., Nov. 8, 2012, “Method and Apparatus for the Treatment of Large and Giant Vascular Defects”) discloses the deployment of multiple permeable shell devices within a single vascular defect. U.S. Pat. No. 11,179,159 (Cox et al., Nov. 23, 2021, “Methods and Devices for Treatment of Vascular Defects”) discloses a device comprising a first hub, a second hub, a support structure having a longitudinal axis, the support structure disposed between the first hub and the second hub, the support structure including a plurality of struts, and a layer of material disposed over the plurality of struts, wherein the first hub is cylindrical and connected to an end of each of the struts of the plurality of struts. U.S. patent application 20230107778 (Cox et al., Apr. 6, 2023, “Methods and Devices for Treatment of Vascular Defects”) discloses an expandable body comprising a plurality of elongate filamentary elements each having a first end and a second end, wherein the elements extend from a first end of the device to a second end of the device and back to the first end of the device. U.S. patent application 20110022149 (Cox et al., Jan. 27, 2011, “Methods and Devices for Treatment of Vascular Defects”) discloses a device with first ends secured to a first ring and second ends secured to a second ring with the first and second rings being disposed substantially concentric to the longitudinal axis.

U.S. patent application 20200289125 (Dholakia et al., Sep. 17, 2020, “Filamentary Devices Having a Flexible Joint for Treatment of Vascular Defects”) discloses an implant with a first permeable shell having a proximal end with a concave or recessed section and a second permeable shell having a convex section that mates with the concave or recessed section. U.S. patent application 20210282785 (Dholakia et al., Sep. 16, 2021, “Devices Having Multiple Permeable Shells for Treatment of Vascular Defects”) a device with a plurality of permeable shells connected by a plurality of coils.

U.S. patent applications 20140135811 (Divino et al., May 15, 2014, “Occlusive Devices”), 20140135812 (Divino et al., May 15, 2014, “Occlusive Devices”), 20190282242 (Divino et al., Sep. 19, 2019, “Occlusive Devices”), and 20190290286 (Divino et al., Sep. 26, 2019, “Occlusive Devices”) and also U.S. Pat. No. 10,327,781 (Divino et al., Jun. 25, 2019, “Occlusive Devices”) disclose multiple expandable structures, wherein each of the expandable structures has a unique shape or porosity profile. U.S. patent application 20170281194 (Divino et al., Oct. 5, 2017, “Embolic Medical Devices”) discloses an occlusive device with an elongate member having opposing first and second side edges which extend longitudinally along the member and a member width, wherein this member has a collapsed configuration in which the first and second side edges are curled toward each other about a longitudinal axis of the member. U.S. patent application 20190343532 (Divino et al., Nov. 14, 2019, “Occlusive Devices”) discloses a device with at least one expandable structure which is adapted to transition from a compressed configuration to an expanded configuration when released into an aneurysm.

U.S. patent application 20200155333 (Franano et al., May 21, 2020, “Ballstent Device and Methods of Use”) discloses a rounded, thin-walled, expandable metal structure and a flexible, elongated delivery device. U.S. Pat. No. 11,013,516 (Franano et al., May 25, 2021, “Expandable Body Device and Method of Use”) discloses a single-lobed, thin-walled, expandable body comprising gold, platinum, or silver. U.S. Pat. No. 11,033,275 (Franano et al., Jun. 15, 2021, “Expandable Body Device and Method of Use”) discloses devices, designs, methods of manufacturing and using hollow gold structures that can be folded, wrapped, and compressed. U.S. patent application 20210275187 (Franano et al., Sep. 9, 2021, “Expandable Body Device and Method of Use”) discloses medical devices comprising a single-lobed, thin-walled, expandable body. U.S. patent application 20190053811 (Garza et al., Feb. 21, 2019, “Flow Attenuation Device”) and U.S. Pat. No. 11,071,551 (Garza et al., Jul. 27, 2021, “Flow Attenuation Device”) disclose an embolic device for treating aneurysms with a desired porosity only at discrete sections.

U.S. patent application 20190365385 (Gorochow et al., Dec. 5, 2019, “Aneurysm Device and Delivery System”) and U.S. Pat. No. 10,939,915 (Gorochow et al., Mar. 9, 2021, “Aneurysm Device and Delivery System”) discloses a braid, wherein translating the braid causes a delivery portion to expand and form a distal sack as well as invert into itself. U.S. patent application 20200113576 (Gorochow et al., Apr. 16, 2020, “Folded Aneurysm Treatment Device and Delivery Method”) and U.S. patent application 20210196284 (Gorochow et al., Jul. 1, 2021, “Folded Aneurysm Treatment Device and Delivery Method”) disclose an implant having a braided section that folds to form an outer occlusive sack extending across a neck of an aneurysm to engage a wall of the aneurysm from within a sac of the aneurysm and an inner occlusive sack forming a trough nested within the outer occlusive sack. The implant can be closed at one or more of the braid ends to define a substantially enclosed bowl-shaped volume.

U.S. Pat. No. 10,653,425 (Gorochow et al., May 19, 2020, “Layered Braided Aneurysm Treatment Device”), U.S. patent application 20200367893 (Xu et al., Nov. 26, 2020, “Layered Braided Aneurysm Treatment Device”), U.S. patent application 20200367898 (Gorochow et al., Nov. 26, 2020, “Layered Braided Aneurysm Treatment Device”), U.S. Pat. No. 11,413,046 (Xu et al., Aug. 16, 2022, “Layered Braided Aneurysm Treatment Device”), and U.S. patent application 20200367900 (Pedroso et al., Nov. 26, 2020, “Layered Braided Aneurysm Treatment Device With Corrugations”) disclose a tubular braid comprising an open end, a pinched end, and a predetermined shape; wherein, in the predetermined shape, the tubular braid comprises: a first segment extending from the open end to a first inversion, a second segment encircled by the open end such that the second segment is only partially surrounded by the first segment and extending from the first inversion to a second inversion, and a third segment surrounded by the second segment and extending from the second inversion to the pinched end.

U.S. patent application 20210085333 (Gorochow et al., Mar. 25, 2021, “Inverting Braided Aneurysm Treatment System and Method”), U.S. Pat. No. 11,278,292 (Gorochow et al., Mar. 22, 2022, “Inverting Braided Aneurysm Treatment System and Method”), and U.S. patent application 20220104829 (Gorochow et al., Apr. 7, 2022, “Inverting Braided Aneurysm Treatment System and Method”) disclose a tubular braid with an intrasaccular section, an intravascular section, a pinched section, and a predetermined shape. U.S. patent application 20210169495 (Gorochow et al., Jun. 10, 2021, “Intrasaccular Inverting Braid with Highly Flexible Fill Material”) and U.S. Pat. No. 11,602,350 (Gorochow et al., Mar. 14, 2023, “Intrasaccular Inverting Braid with Highly Flexible Fill Material”) disclose a tubular braided implant which is delivered as a single layer braid, inverted into itself during deployment to form at least two nested sacks and includes additional braid material that can fill the innermost sack.

U.S. patent application 20210186518 (Gorochow et al., Jun. 24, 2021, “Implant Having an Intrasaccular Section and Intravascular Section”) and U.S. Pat. No. 11,457,926 (Gorochow et al., Oct. 4, 2022, “Implant Having an Intrasaccular Section and Intravascular Section”) disclose a tubular braid with an intrasaccular section, an intravascular section, a pinched section, and a predetermined shape. U.S. Pat. No. 11,058,430 (Gorochow et al., Jul. 13, 2021, “Aneurysm Device and Delivery System”) discloses a braid with a proximal expandable portion for positioning inside an aneurysm and sealing across the neck of the aneurysm. U.S. Pat. No. 11,076,861 (Gorochow et al., Aug. 3, 2021, “Folded Aneurysm Treatment Device and Delivery Method”) discloses an implant with a fold which defines an annular ridge and a radiopaque marker band. U.S. patent application 20210106338 (Gorochow, Apr. 15, 2021, “Spiral Delivery System for Embolic Braid”) discloses a braided implant having a spiral segment. U.S. patent application 20210145449 (Gorochow, May 20, 2021, “Implant Delivery System with Braid Cup Formation”) discloses an implant system with an engagement wire, a pull wire, and a braided implant having a distal ring thereon. U.S. patent application 20210169498 (Gorochow, Jun. 10, 2021, “Delivery of Embolic Braid”) discloses a braided implant delivery system which attaches a braided implant having a band to a delivery tube, positions the braided implant within an aneurysm, and then releases the band from the delivery tube. U.S. Pat. No. 11,051,825 (Gorochow, Jul. 6, 2021, “Delivery System for Embolic Braid”) discloses a braided implant which is attached to a releasing component that can be detachably engaged with a delivery tube and a pull wire.

U.S. patent application 20210338250 (Gorochow et al., Nov. 4, 2021, “Intrasaccular Flow Diverter”) and U.S. Pat. No. 11,523,831 (Gorochow et al., Dec. 13, 2022, “Intrasaccular Flow Diverter”) disclose an interior fill braid physically which is inverted over itself to form a proximal inverted end and an opposite free end and a dome braid disposed distally of and secured to the interior fill braid. U.S. patent application 20220202425 (Gorochow et al., Jun. 30, 2022, “Semispherical Braided Aneurysm Treatment System and Method”) discloses a tubular braid with three segments and two inversions, one of the three segments extending between the two inversions and forming a sack. U.S. patent application 20210338247 (Gorochow, Nov. 4, 2021, “Double Layer Braid”) discloses a double layered braid for treating an aneurysm. U.S. Pat. No. 11,583,282 (Gorochow et al., Feb. 21, 2023, “Layered Braided Aneurysm Treatment Device”) discloses a method for shaping a tubular braid into a predetermined shape. U.S. patent application 20180070955 (Greene et al., Mar. 15, 2018, “Embolic Containment”) discloses a method of treating a neurovascular arteriovenous malformation with liquid embolic and dimethyl sulfoxide.

U.S. patent applications 20150313605 (Griffin, Nov. 5, 2015, “Occlusion Device”) and 20190059909 (Griffin, Feb. 28, 2019, “Occlusion Device”) and also U.S. Pat. No. 10,130,372 (Griffin, Nov. 20, 2018, “Occlusion Device”) and U.S. Pat. No. 11,389,174 (Griffin, Jul. 19, 2022, “Occlusion Device”) disclose an occlusion device with a substantially solid marker having a proximal end, and a distal end; and a low profile resilient mesh body attached to the distal end of the marker. U.S. patent application 20170156734 (Griffin, Jun. 8, 2017, “Occlusion Device”), U.S. Pat. No. 10,285,711 (Griffin, May 14, 2019, “Occlusion Device”), U.S. patent application 20190269414 (Griffin, Sep. 5, 2019, “Occlusion Device”), U.S. patent application 20210153871 (Griffin, May 27, 2021, “Occlusion Device”), and U.S. patent application 20220313274 (Griffin, Oct. 6, 2022, “Occlusion Device”) disclose a continuous compressible mesh structure comprising axial mesh carriages configured end to end, wherein each end of each carriage is a pinch point in the continuous mesh structure. U.S. Pat. No. 11,471,162 (Griffin, Oct. 18, 2022, “Occlusion Device”) discloses an occlusion device for implantation into a body lumen or aneurysm which has a continuous compressible mesh structure comprising axial mesh carriages configured end to end, wherein each end of each carriage is a pinch point in the continuous mesh structure.

U.S. patent application 20200187953 (Hamel et al., Jun. 18, 2020, “Devices, Systems, and Methods for the Treatment of Vascular Defects”) discloses a mesh comprising a first end portion, a second end portion, and a length extending between the first and second end portions, and a first lateral edge, a second lateral edge, and a width extending between the first and second lateral edges. U.S. patent application 20210353299 (Hamel et al., Nov. 18, 2021, “Devices, Systems, and Methods for the Treatment of Vascular Defects”) discloses a mesh that is curved along its length with an undulating contour across at least a portion of one or both of its length or its width. U.S. patent application 20220330947 (Henkes et al., Oct. 20, 2022, “Implant for the Treatment of Aneurysms”) discloses an implant which is rolled up relative to a radial axis in order to form a balled-up configuration.

U.S. patent application 20140358178 (Hewitt et al., Dec. 4, 2014, “Filamentary Devices for Treatment of Vascular Defects”), U.S. Pat. No. 9,078,658 (Hewitt et al., Jul. 14, 2015, “Filamentary Devices for Treatment of Vascular Defects”), U.S. patent application 20160249934 (Hewitt et al., Sep. 1, 2016, “Filamentary Devices for Treatment of Vascular Defects”), U.S. Pat. No. 9,955,976 (Hewitt et al., May 1, 2018, “Filamentary Devices for Treatment of Vascular Defects”), U.S. patent application 20180206849 (Hewitt et al., Jul. 26, 2018, “Filamentary Devices for the Treatment of Vascular Defects”), U.S. patent application 20210007754 (Milhous et al., Jan. 14, 2021, “Filamentary Devices for Treatment of Vascular Defects”), U.S. Pat. No. 10,939,914 (Hewitt et al., Mar. 9, 2021, “Filamentary Devices for the Treatment of Vascular Defects”), and U.S. patent application 20210275184 (Hewitt et al., Sep. 9, 2021, “Filamentary Devices for Treatment of Vascular Defects”) disclose occlusion devices with permeable shells made of woven braided mesh having a variable mesh density and/or porosity. U.S. patent application 20160249935 (Hewitt et al., Sep. 1, 2016, “Devices for Therapeutic Vascular Procedures”), U.S. patent application 20160367260 (Hewitt et al., Dec. 22, 2016, “Devices for Therapeutic Vascular Procedures”), U.S. Pat. No. 9,629,635 (Hewitt et al., Apr. 25, 2017, “Devices for Therapeutic Vascular Procedures”), and U.S. patent application 20170128077 (Hewitt et al., May 11, 2017, “Devices for Therapeutic Vascular Procedures”) disclose a self-expanding resilient permeable shell and a metallic coil secured to the distal end of the permeable shell.

U.S. Pat. No. 9,492,174 (Hewitt et al., Nov. 15, 2016, “Filamentary Devices for Treatment of Vascular Defects”), U.S. patent application 20170095254 (Hewitt et al., Apr. 6, 2017, “Filamentary Devices for Treatment of Vascular Defects”), U.S. Pat. No. 10,136,896 (Hewitt et al., Nov. 27, 2018, “Filamentary Devices for Treatment of Vascular Defects”), U.S. patent application 20190192166 (Hewitt et al., Jun. 27, 2019, “Filamentary Devices for Treatment of Vascular Defects”), U.S. patent application 20200289124 (Rangwala et al., Sep. 17, 2020, “Filamentary Devices for Treatment of Vascular Defects”), U.S. Pat. No. 10,813,645 (Hewitt et al., Oct. 27, 2020, “Filamentary Devices for Treatment of Vascular Defects”), and U.S. patent application 20210106337 (Hewitt et al., Apr. 15, 2021, “Filamentary Devices for Treatment of Vascular Defects”) disclose a self-expanding permeable shell having a radially constrained elongated state configured for delivery within a catheter lumen, an expanded state with a globular and longitudinally shortened configuration relative to the radially constrained state, and a plurality of elongate filaments that are woven together.

U.S. patent application 20190223881 (Hewitt et al., Jul. 25, 2019, “Devices for Therapeutic Vascular Procedures”) discloses a self-expanding resilient permeable shell made from elongate resilient filaments with a distal region that extends beyond the distal end of the permeable shell. U.S. patent application 20200289126 (Hewitt et al., Sep. 17, 2020, “Filamentary Devices for Treatment of Vascular Defects”), U.S. Pat. No. 11,317,921 (Hewitt et al., May 3, 2022, “Filamentary Devices for Treatment of Vascular Defects”), and U.S. patent application 20220257258 (Hewitt et al., Aug. 18, 2022, “Filamentary Devices for Treatment of Vascular Defects”) disclose a permeable shell or mesh with a stiffer proximal portion at the neck of an aneurysm. U.S. patent application 20220192678 (Hewitt et al., Jun. 23, 2022, “Filamentary Devices for Treatment of Vascular Defects”) discloses an implant having a first permeable shell having a proximal hub and an open distal end and a second permeable shell having a distal hub and an open proximal end. U.S. patent application 20220257260 (Hewitt et al., Aug. 18, 2022, “Filamentary Devices for Treatment of Vascular Defects”) discloses an implant having multiple mesh layers. U.S. patent application 20230114169 (Hewitt et al., Apr. 13, 2023, “Devices for Treatment of Vascular Defects”) discloses a permeable woven implant with a radially-constrained state for delivery within a catheter and an expanded state thereafter.

U.S. patent application 20230039246 (Hossan et al., Feb. 9, 2023, “Non-Braided Biodegradable Flow Diverting Device for Endovascular Treatment of Aneurysm and Associated Fabrication Method”) discloses a biodegradable flow-diverting device that regulates blood flow into an aneurysmal sac. U.S. patent application 20230263528 (Jones, Aug. 24, 2023, “Intrasaccular Flow Diverter and Related Methods”) discloses an intrasaccular flow diverter with a plurality of wires which are coiled to form a collapsible, substantially spherical frame. U.S. patent application 20230252631 (Kashyap et al., Aug. 10, 2023, “Neural Network Apparatus for Identification, Segmentation, and Treatment Outcome Prediction for Aneurysms”) discloses using using medical imaging and a neural network to predict outcomes from the potential use of one or more different intrasaccular implant devices. U.S. patent application 20210353300 (Kottenmeier et al., Nov. 18, 2021, “Systems and Methods for Treatment of Defects in the Vasculature”) discloses aneurysm occlusion methods and systems including an expandable stent.

U.S. patent application 20230225735 (Kulak et al., Jul. 20, 2023, “Expandable Devices for Treating Body Lumens”) discloses a tubular mesh which curves along its longitudinal dimension when implanted in an aneurysm cavity. U.S. patent application 20210137526 (Lee et al., May 13, 2021, “Embolic Devices for Occluding Body Lumens”) discloses an embolic device with a first segment forming a first three-dimensional structure, wherein the first three-dimensional structure defines a cavity; and a second segment forming a second three-dimensional structure; wherein the cavity of the first three-dimensional structure is configured to accommodate at least a majority of the second three-dimensional structure. U.S. patent application 20230017150 (Lee et al., Jan. 19, 2023, “Hydrogel Stent and Embolization Device for Cerebral Aneurysm”) discloses a hydrogel stent for occluding a cerebral aneurysm.

U.S. patent application 20210128169 (Li et al., May 6, 2021, “Devices, Systems, and Methods for Treatment of Intracranial Aneurysms”) and U.S. patent application 20210153872 (Nguyen et al., May 27, 2021, “Devices, Systems, and Methods for Treatment of Intracranial Aneurysms”) disclose delivering an occlusive member to an aneurysm cavity and deforming a shape of the occlusive member via introduction of an embolic element to a space between the occlusive member and an inner surface of the aneurysm wall. U.S. patent application 20210128160 (Li et al., May 6, 2021, “Systems and Methods for Treating Aneurysms”), U.S. patent application 20210128167 (Patel et al., May 6, 2021, “Systems and Methods for Treating Aneurysms”), U.S. patent application 20210128168 (Nguyen et al., May 6, 2021, “Systems and Methods for Treating Aneurysms”) and disclose delivering an occlusive member (e.g., an expandable braid) to an aneurysm sac in conjunction with an embolic element (e.g., coils, embolic material).

U.S. patent application 20190192168 (Lorenzo et al., Jun. 27, 2019, “Aneurysm Device and Delivery Method”) and U.S. Pat. No. 10,716,574 (Lorenzo et al., Jul. 21, 2020, “Aneurysm Device and Delivery Method”) discloses a self-expanding braid for treating an aneurysm, including a method for inverting and buckling a proximal segment. U.S. patent application 20190223878 (Lorenzo et al., Jul. 25, 2019, “Aneurysm Device and Delivery System”) and U.S. patent application 20200397447 (Lorenzo et al., Dec. 24, 2020, “Aneurysm Device and Delivery System”) discloses an expandable segment which radially expands inside an outer occlusive sack. U.S. patent application 20210007755 (Lorenzo et al., Jan. 14, 2021, “Intrasaccular Aneurysm Treatment Device with Varying Coatings”) discloses an aneurysm intrasaccular implant with coated regions. U.S. Pat. No. 10,905,430 (Lorenzo et al., Feb. 2, 2021, “Aneurysm Device and Delivery System”) discloses an expandable segment which radially expands inside an outer occlusive sack.

U.S. patent applications 20150272589 (Lorenzo, Oct. 1, 2015, “Aneurysm Occlusion Device”) and 20190008522 (Lorenzo, Jan. 10, 2019, “Aneurysm Occlusion Device”) and also U.S. Pat. No. 11,076,860 (Lorenzo, Aug. 3, 2021, “Aneurysm Occlusion Device”) disclose a tubular structure which is constrained by a control ring. U.S. patent application 20180242979 (Lorenzo, Aug. 30, 2018, “Aneurysm Device and Delivery System”) and U.S. Pat. No. 10,751,066 (Lorenzo, Aug. 25, 2020, “Aneurysm Device and Delivery System”) disclose a self-expanding braided tubular member. U.S. patent application 20200375606 (Lorenzo, Dec. 3, 2020, “Aneurysm Method and System”) discloses a braided implant which is invertible about the distal implant end. U.S. patent application 20210177429 (Lorenzo, Jun. 17, 2021, “Aneurysm Method and System”) discloses a vaso-occlusive device with at least two nested sacks. U.S. Pat. No. 11,123,077 (Lorenzo et al., Sep. 21, 2021, “Intrasaccular Device Positioning and Deployment System”) discloses implant deployment systems including a braided implant that can be detachably attached to a delivery tube by an expansion ring. U.S. patent application 20210330331 (Lorenzo, Oct. 28, 2021, “Aneurysm Occlusion Device”) and U.S. Pat. No. 11,154,302 (Lorenzo et al., Oct. 26, 2021, “Aneurysm Occlusion Device”) disclose an occlusion device with a substantially annular body disposed on the proximal end region of the device. U.S. patent application 20200038034 (Maguire et al., Feb. 6, 2020, “Vessel Occluder”) discloses a vessel occluder with an expandable mesh portion having a flexible membrane that expands within a cavity of the expandable mesh portion.

U.S. patent application 20130245667 (Marchand et al., Sep. 19, 2013, “Filamentary Devices and Treatment of Vascular Defects”) discloses a self-expanding resilient permeable shell wherein filaments are bundled and secured to each other at a proximal end. U.S. patent application 20160249937 (Marchand et al., Sep. 1, 2016, “Multiple Layer Filamentary Devices for Treatment of Vascular Defects”), U.S. Pat. No. 9,918,720 (Marchand et al., Mar. 20, 2018, “Multiple Layer Filamentary Devices for Treatment of Vascular Defects”), and U.S. Pat. No. 10,238,393 (Marchand et al., Mar. 26, 2019, “Multiple Layer Filamentary Devices for Treatment of Vascular Defects”) disclose a permeable shell and an inner structure configured to occlude blood flow. U.S. patent application 20180000489 (Marchand et al., Jan. 4, 2018, “Filamentary Devices for Treatment of Vascular Defects”) discloses a self-expanding resilient permeable shell having a plurality of elongate resilient filaments with a woven structure. U.S. Pat. No. 10,610,231 (Marchand et al., Apr. 7, 2020, “Filamentary Devices for Treatment of Vascular Defects”) discloses a self-expanding resilient permeable shell with a plurality of elongate resilient filaments with a woven structure, wherein the plurality of filaments includes small filaments and large filaments, and wherein the small filaments have a transverse dimension which is smaller than the transverse dimension of the large filaments. U.S. patent application 20200281603 (Marchand et al., Sep. 10, 2020, “Filamentary Devices for Treatment of Vascular Defects”) discloses a permeable shell including a really swell polymer.

U.S. Pat. No. 11,517,321 (Mauger et al., Dec. 6, 2022, “System and Methods for Embolized Occlusion of Neurovascular Aneurysms”) discloses an occlusion device with a reinforcing portion with no porosity. U.S. Pat. No. 11,589,872 (Mauger, Feb. 28, 2023, “Vascular Occlusion Devices Utilizing Thin Film Nitinol Foils”) discloses an implantable occlusion device wherein mesh components are wrapped around a support structure and slot that enables a disc to be wrapped around the support structure with overlapping portions. U.S. patent application 20230285031 (Mayer et al, Sep. 14, 2023, “Device for Restricting Blood Flow to Aneurysms”) discloses a non-occlusive device with a coilable section and a docking section. U.S. patent application 20220249098 (Milhous et al., Aug. 11, 2022, “Filamentary Devices for Treatment of Vascular Defects”) discloses a permeable implant with a plurality of scaffolding filaments. U.S. patent application 20230039773 (Monstadt et al., Feb. 9, 2023, “Implant for Treating Aneurysms”) discloses an implant which is preset to a specific structure.

U.S. Pat. No. 11,426,175 (Morita et al., Aug. 30, 2022, “Expansile Member”) discloses an occlusive system comprising: a catheter; a shell deliverable through the catheter, a delivery pusher detachably connected to the shell and configured to navigate the shell through the catheter, wherein the shell has a globular shaped portion. U.S. patent application 20210129275 (Nguyen et al., May 6, 2021, “Devices, Systems, and Methods for Treating Aneurysms”) discloses methods of manufacturing an occlusive device including conforming a mesh to a forming assembly and setting a shape of the mesh based on the forming assembly. U.S. patent application 20210275779 (Northrop, Sep. 9, 2021, “Actuating Elements for Bending Medical Devices”) discloses an actuating element causes a tube to bend. U.S. Pat. No. 11,498,165 (Patel et al., Nov. 15, 2022, “Systems and Methods for Treating Aneurysms”) discloses an occlusive implant with a conduit which receives a liquid embolic.

U.S. patent application 20210346032 (Patterson et al., Nov. 11, 2021, “Devices for Treatment of Vascular Defects”) discloses an expandable stent for placement in a parent vessel proximal, near, or adjacent an aneurysm. U.S. Pat. No. 11,607,226 (Pedroso et al., Mar. 21, 2023, “Layered Braided Aneurysm Treatment Device with Corrugations”) discloses an implant with a proximal inversion and two segments. U.S. Pat. No. 11,058,431 (Pereira et al., Jul. 13, 2021, “Systems and Methods for Treating Aneurysms”) discloses an inverted mesh tube having an outer layer and an inner layer, wherein the outer layer transitions to the inner layer at an inversion fold located at or adjacent to the distal end of the occlusion element. U.S. patent application 20170258473 (Plaza et al., Sep. 14, 2017, “Systems and Methods for Delivery of Stents and Stent-Like Devices”) discloses an elongate tubular member having a lumen, an expandable stent, and a delivery device which is placed in a cerebral vessel adjacent to an aneurysm.

U.S. patent applications 20060155323 (Porter et al., Jul. 13, 2006, “Intra-Aneurysm Devices”) and 20190298379 (Porter et al., Oct. 3, 2019, “Intra-Aneurysm Devices”) and also U.S. Pat. No. 10,265,075 (Porter et al., Apr. 23, 2019, “Intra-Aneurysm Devices”) disclose an occlusive device having a neck and a dome. U.S. patent application 20210052279 (Porter et al., Feb. 25, 2021, “Intra-Aneurysm Devices”) discloses a device including an upper member that sits against the dome of an aneurysm, a lower member that sits in the neck of the aneurysm, and a means of adjusting the overall dimensions of the device. U.S. patent application 20210128165 (Pulugurtha et al., May 6, 2021, “Systems and Methods for Treating Aneurysms”) and U.S. Pat. No. 11,305,387 (Pulugurtha et al., Apr. 19, 2022, “Systems and Methods for Treating Aneurysms”) disclose a distal conduit coupled to an occlusive member with a first lumen extending therethrough and a proximal conduit with a second lumen extending therethrough.

U.S. patent application 20220039804 (Rangwala et al., Feb. 10, 2022, “Flow-Diverting Implant and Delivery Method”) discloses a saddle-shaped braided mesh diverter that covers the neck of an aneurysm. U.S. Pat. No. 11,559,309 (Rangwala et al., Jan. 24, 2023, “Filamentary Devices for Treatment of Vascular Defects”) discloses a permeable implant whose proximal portion is stiffer. U.S. patent application 20230277184 (Rashidi et al., Sep. 7, 2023, “Occlusive Devices with Thrombogenic Inserts”) discloses an expandable mesh which spans a neck of the aneurysm with an insert configured to promote thrombosis.

U.S. patent application 20170079662 (Rhee et al., Mar. 23, 2017, “Occlusive Devices”) discloses an implant with a frame and a mesh component, wherein the mesh component has a first porosity and the frame has a second porosity. U.S. Pat. No. 10,478,194 (Rhee et al., Nov. 19, 2019, “Occlusive Devices”) and U.S. patent application 20200038032 (Rhee et al., Feb. 6, 2020, “Occlusive Devices”) disclose an implant with a frame and a mesh component, wherein the mesh component has a first porosity and the frame has a second porosity. U.S. patent application 20210128162 (Rhee et al., May 6, 2021, “Devices, Systems, and Methods for Treatment of Intracranial Aneurysms”) discloses introduction of an embolic element to a space between an occlusive member and an inner surface of the aneurysm wall.

U.S. patent application 20140330299 (Rosenbluth et al., Nov. 6, 2014, “Embolic Occlusion Device and Method”), U.S. patent application 20180303486 (Rosenbluth et al., Oct. 25, 2018, “Embolic Occlusion Device and Method”), and U.S. patent application 20210259699 (Rosenbluth et al., Aug. 26, 2021, “Embolic Occlusion Device and Method”) disclose an occlusion device with a tubular braided member having a first end and a second end and extending along a longitudinal axis, the tubular braided member having a repeating pattern of larger diameter portions and smaller diameter portions arrayed along the longitudinal axis. U.S. patent applications 20160022445 (Ruvalcaba et al., Jan. 28, 2016, “Occlusive Device”) and 20190343664 (Ruvalcaba et al., Nov. 14, 2019, “Occlusive Device”) U.S. Pat. No. 11,389,309 (Ruvalcaba et al., Jul. 19, 2022, “Occlusive Device”) disclose an aneurysm embolization device having a single, continuous piece of material that is shape set into a plurality of distinct structural components.

U.S. patent application 20020169473 (Sepetka et al., Nov. 14, 2002, “Devices and Methods for Treating Vascular Malformations”) discloses a device with a primary coil to provide structural integrity and secondary windings to fill interstitial spaces. U.S. patent application 20080281350 (Sepetka et al., Nov. 13, 2008, “Aneurysm Occlusion Devices”) discloses an implantable occlusion device with a concave or cup-shaped shape after implantation. U.S. Pat. No. 8,597,320 (Sepetka et al., Dec. 3, 2013, “Devices and Methods for Treating Vascular Malformations”) discloses a method of filling an aneurysm by advancing a device with a proximal collar and a distal collar through a vascular system and then positioning the device within an aneurysm. U.S. patent application 20140200607 (Sepetka et al., Jul. 17, 2014, “Occlusive Device”), U.S. patent application 20190274691 (Sepetka et al., Sep. 12, 2019, “Occlusive Device”), and U.S. Pat. No. 11,045,203 (Sepetka et al., Jun. 29, 2021, “Occlusive Device”) disclose multiple sequentially deployed occlusive devices that are connected together to create an extended length. U.S. patent application 20210282784 (Sepetka et al., Sep. 16, 2021, “Occlusive Device”) discloses a device comprising a plurality of braided wires and an embolic coil.

U.S. patent application 20170224350 (Shimizu et al., Aug. 10, 2017, “Devices for Vascular Occlusion”), U.S. Pat. No. 10,729,447 (Shimizu et al., Aug. 4, 2020, “Devices for Vascular Occlusion”), U.S. patent application 20200323534 (Shimizu et al., Oct. 15, 2020, “Devices for Vascular Occlusion”), U.S. Pat. No. 10,980,545 (Bowman et al., Apr. 20, 2021, “Devices for Vascular Occlusion”), U.S. patent application 20210228214 (Bowman et al., Jul. 29, 2021, “Devices for Vascular Occlusion”), and U.S. patent application 20210228214 (Bowman et al., Jul. 29, 2021, “Devices for Vascular Occlusion”) disclose an occlusive device, an occlusive device delivery system, method of using, method of delivering an occlusive device, and method of making an occlusive device to treat various intravascular conditions. U.S. patent application 20230031965 (Sivapatham, Feb. 2, 2023, “Intrasaccular Stent Device for Aneurysm Treatment”) discloses a system for treating an aneurysm in a blood vessel comprising a catheter, a guidewire, a delivery wire, an intrasaccular stent/retaining device removably attached to the delivery wire, and endovascular coiling.

U.S. patent application 20200305886 (Soto Del Valle et al, Oct. 1, 2020, “Aneurysm Treatment Device”) and U.S. patent application 20220225997 (Soto Del Valle et al., Jul. 21, 2022, “Aneurysm Treatment Device”) disclose a device with an expandable sack with a free open end and an elongated looping portion. U.S. patent application 20200305885 (Soto Del Valle et al, Oct. 1, 2020, “Aneurysm Treatment Device”) discloses an occlusion device that expands to form a cup shape within an aneurysm sac. U.S. patent application 20200375607 (Soto Del Valle et al., Dec. 3, 2020, “Aneurysm Device and Delivery System”) discloses a method of expanding mesh segments to form an outer occlusive sack and an inner occlusive sack. U.S. Pat. No. 11,337,706 (Soto Del Valle et al., May 24, 2022, “Aneurysm Treatment Device”) discloses an implant having an elongated portion and an expandable braided sack portion.

U.S. patent application 20210282789 (Vu et al., Sep. 16, 2021, “Multiple Layer Devices for Treatment of Vascular Defects”) discloses a first permeable shell and a second permeable shell, where the second permeable shell sits within an interior cavity of the first permeable shell. U.S. patent application 20220175389 (Wallace et al., Jun. 9, 2022, “Vaso-Occlusive Devices Including a Friction Element”) discloses a vaso-occlusive implant with a friction element between a soft braided member and a coil. U.S. patent application 20200187952 (Walsh et al., Jun. 18, 2020, “Intrasaccular Flow Diverter for Treating Cerebral Aneurysms”) and U.S. patent application 20220151632 (Walsh et al., May 19, 2022, “Intrasaccular Flow Diverter for Treating Cerebral Aneurysms”) disclose a stabilizing frame with two parts, the first part sized to anchor within the sac of the aneurysm and the exterior part sized to anchor against a region of the blood vessel wall adjacent the aneurysm neck.

U.S. patent application 20200029973 (Walzman, Jan. 30, 2020, “Mash Cap for Ameliorating Outpouchings”) discloses an embolic device comprising a control element, a catheter element, a delivery microcatheter hypotube, a detachment element, a mesh disc, a distal opening, and at least one attached extension arm. U.S. patent application 20200405347 (Walzman, Dec. 31, 2020, “Mesh Cap for Ameliorating Outpouchings”) discloses a self-expandable occluding device which covers the neck of an outpouching and serves as a permanent embolic plug. U.S. Pat. No. 10,398,441 (Warner et al., Sep. 3, 2019, “Vascular Occlusion”) discloses a vascular treatment system with a containment device, a pusher, and a stopper ring. U.S. Pat. No. 11,166,731 (Wolfe et al., Nov. 9, 2021, “Systems and Methods for Treating Aneurysms”) discloses an inverted mesh tube having an outer layer and an inner layer, the outer layer transitioning to the inner layer at an inversion fold.

U.S. patent application 20200367906 (Xu et al., Nov. 26, 2020, “Aneurysm Treatment With Pushable Ball Segment”) and U.S. patent application 20230016312 (Xu et al., Jan. 19, 2023, “Aneurysm Treatment with Pushable Implanted Braid”) disclose a braided implant with a retractable dual proximal layer. U.S. patent application 20220087681 (Xu et al., Mar. 24, 2022, “Inverting Braided Aneurysm Implant with Dome Feature”) discloses an implant with a dome feature configured to press into aneurysm walls near the aneurysm's dome and facilitate securement of the braid across the aneurysm's neck. U.S. Pat. No. 11,497,504 (Xu et al., Nov. 15, 2022, “Aneurysm Treatment with Pushable Implanted Braid”) discloses a braided implant with a retractable dual proximal layer. U.S. Pat. No. 11,596,412 (Xu et al., Mar. 7, 2023, “Aneurysm Device and Delivery System”) discloses a braid with a proximal portion which goes across an aneurysm neck and an expandable distal portion. U.S. patent application 20230061363 (Yee et al., Mar. 2, 2023, “Embolic Device with Improved Neck Coverage”) discloses an embolic device with a flexible structure which has a series of alternating narrow portions and link portions.

U.S. patent application 20200367896 (Zaidat et al., Nov. 26, 2020, “Systems and Methods for Treating Aneurysms”) discloses an apparatus for treating an aneurysm in a blood vessel with a first tubular mesh having a first end and a second end coupled together at a proximal end of the occlusion element. U.S. Pat. No. 11,202,636 (Zaidat et al., Dec. 21, 2021, “Systems and Methods for Treating Aneurysms”), U.S. patent application 20220022884 (Wolfe et al., Jan. 27, 2022, “Systems and Methods for Treating Aneurysms”), and U.S. patent application 20220211383 (Pereira et al., Jul. 7, 2022, “Systems and Methods for Treating Aneurysms”) disclose an apparatus for treating an aneurysm including an occlusion element configured to be releasably coupled to an elongate delivery shaft and a distal end, a proximal end, and a longitudinal axis extending between the distal end and the proximal end. U.S. patent application 20220054141 (Zaidat et al., Feb. 24, 2022, “Systems and Methods for Treating Aneurysms”) discloses an apparatus for treating an aneurysm in a blood vessel with a first tubular mesh having a first end and a second end coupled together at a proximal end of the occlusion element.

### SUMMARY OF THE INVENTION

This invention can be embodied in an intrasaccular device or system for occluding a cerebral aneurysm which includes: a proximal neck bridge which expands within an aneurysm sac to span the aneurysm neck; a distal net or mesh which is expanded within the aneurysm sac between the proximal neck bridge and the aneurysm dome; and embolic members which are inserted into the net or mesh, wherein insertion of the embolic members expands the net or mesh.

This invention can also be embodied in a method for intrasaccular occlusion of a cerebral aneurysm which includes: inserting a guidewire into an aneurysm sac; inserting a catheter into the aneurysm sac; inserting a distal flexible net or mesh through the catheter into the aneurysm sac; inserting a proximal neck bridge through the catheter into the aneurysm sac between the net or mesh and the aneurysm neck, wherein the neck bridge expands in the aneurysm sac after it exits the catheter; and inserting embolic members through the catheter into the net or mesh, wherein accumulation of the embolic members in the net or mesh expands the net or mesh.

### BRIEF INTRODUCTION TO THE FIGURES

FIGS. 1 through 3 show three sequential views of how a single-layer bowl-shaped proximal neck bridge can be formed from a tubular mesh.

FIGS. 4 through 8 show five sequential views of how a double-layer bowl-shaped proximal neck bridge can be formed from a tubular mesh.

FIG. 9 shows an example of a honeycomb distal net or mesh made by 3D printing.

FIG. 10 shows an example of a globular distal net or mesh from a tubular woven or braided mesh.

FIG. 11 shows an example of a distal net or mesh made from a compliant balloon with holes.

FIGS. 12 and 13 show two sequential views of a proximal neck bridge and a distal net or mesh which are connected and nested before insertion into an aneurysm sac.

FIGS. 14 through 16 show three sequential views of a proximal neck bridge and a distal net or mesh which are connected and nested after insertion into an aneurysm sac.

FIG. 17 shows an example of “string-of-pearl” longitudinal strands of embolic pieces being inserted into a distal net or mesh.

FIG. 18 shows an example of separate embolic pieces being inserted into a distal net or mesh.

FIG. 19 shows an example of embolic coils being inserted into a distal net or mesh.

FIGS. 20 through 22 show three sequential views of a distal net or mesh, a proximal neck bridge, and embolic members being deployed to occlude an aneurysm, wherein FIG. 20 shows the net or mesh and neck bridge before they are connected and nested, FIG. 21 shows them after they are connected and nested, and FIG. 22 shows “string-of-pearl” longitudinal strands of embolic pieces being inserted into the net or mesh.

### DETAILED DESCRIPTION OF THE FIGURES

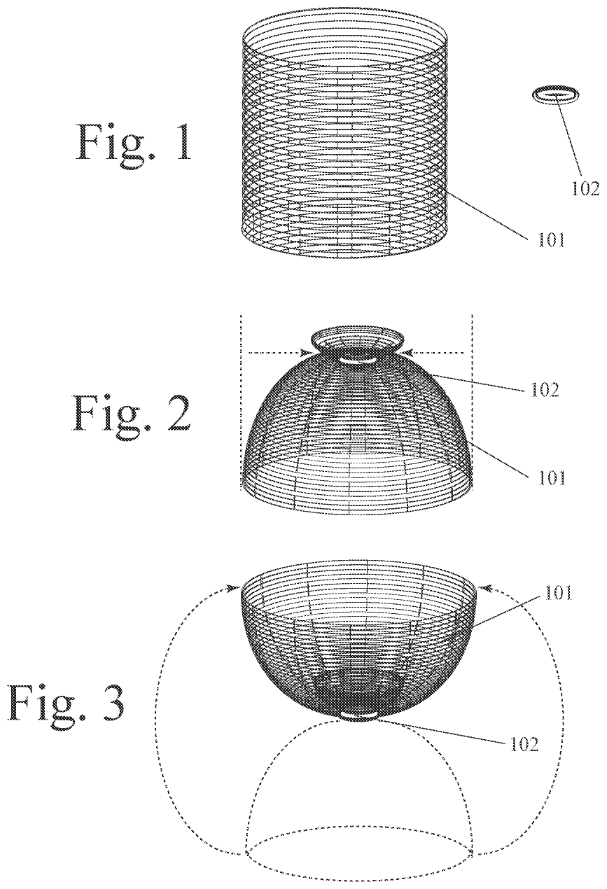

Before discussing the specific embodiments of this invention which are shown in FIGS. 1 through 22, this disclosure provides an introductory section which covers some of the general concepts, components, and methods which comprise this invention. Where relevant, these concepts, components, and methods can be applied as variations to the examples shown in FIGS. 1 through 22 which are discussed afterwards.

In an example, a distal net or mesh can be used in combination with a proximal neck bridge to comprise an intrasaccular system or device for occluding a cerebral aneurysm. In an example, an intrasaccular device or system for occluding a cerebral aneurysm can comprise: a guidewire which is inserted into an aneurysm sac; at least one catheter which is advanced along the guidewire into the aneurysm sac; a proximal neck bridge which is delivered through the at least one catheter, expands within an aneurysm sac, and spans the aneurysm neck; a distal flexible net or mesh which is delivered through the at least one catheter and expanded within the aneurysm sac between the proximal neck bridge and the aneurysm dome; and embolic members which are inserted into the net or mesh, wherein insertion of the embolic members expands the net or mesh.

In an example, an intrasaccular device or system for occluding a cerebral aneurysm can comprise: a distal flexible net or mesh, wherein the net or mesh is inserted into an aneurysm sac, wherein the net or mesh is expanded in the aneurysm sac by insertion of embolic members into the net or mesh, and wherein there is a first portion of a connection mechanism on the net or mesh; and a proximal neck bridge, wherein the neck bridge is inserted into the aneurysm sac, wherein the neck bridge is between the net or mesh and the aneurysm neck, wherein the neck bridge expands within the aneurysm sac to span the aneurysm neck, wherein the neck bridge has a proximal hole or opening through which embolic members are inserted into the net or mesh, wherein there is a second portion of the connection mechanism on the neck bridge, and wherein the first portion of the connection mechanism and the second portion of the connection mechanism fit, snap, clip, latch, plug, stick, adhere, or interlock together, thereby connecting the net or mesh and the neck bridge to each other.

In an example, a method for intrasaccular occlusion of a cerebral aneurysm can comprise: inserting a guidewire into an aneurysm sac; inserting a catheter into the aneurysm sac; inserting a distal flexible net or mesh through the catheter into the aneurysm sac; inserting a proximal neck bridge through the catheter into the aneurysm sac between the net or mesh and the aneurysm neck, wherein the neck bridge expands in the aneurysm sac after it exits the catheter; inserting embolic members through the catheter into the net or mesh, wherein accumulation of the embolic members in the net or mesh expands the net or mesh; and withdrawing the catheter from the net or mesh and the neck bridge.

In an example, a system and device for occluding a cerebral aneurysm can comprise: a flexible distal net or mesh which is inserted into an aneurysm sac; a proximal neck bridge which is inserted into the aneurysm sac between the distal net or mesh and the aneurysm neck; embolic members which are inserted into the distal net or mesh; a first annular member (e.g. ring, band, washer, circular wire, tie, cylinder, string, or tube) which is attached to the distal net or mesh; a second annular member (e.g. ring, band, washer, circular wire, tie, cylinder, string, or tube) attached to the proximal neck bridge; a catheter through which embolic members are inserted into the distal net or mesh; a first wire which pushes the distal net or mesh through a catheter into an aneurysm sac; and a second wire which pushes the proximal neck bridge through the catheter into the aneurysm sac.

In an example, an intrasaccular device or system for occluding a cerebral aneurysm can comprise: a proximal neck bridge which expands within an aneurysm sac and spans the aneurysm neck; a distal flexible net or mesh which is expanded within the aneurysm sac between the proximal neck bridge and the aneurysm dome; embolic members which are inserted into the distal net or mesh, wherein insertion of the embolic members expands the distal flexible net or mesh; a guidewire; and one or more catheters, wherein the guidewire is inserted into the aneurysm sac to guide the one or more catheters into the aneurysm sac, and wherein the neck bridge, net or mesh, and embolic members are delivered to the aneurysm sac through the one or more catheters.

In an example, a method for occluding an aneurysm can comprise: making a distal flexible net or mesh; and making a proximal neck bridge; wherein the distal flexible net or mesh is configured to be inserted through a catheter into an aneurysm sac; wherein the proximal neck bridge is configured to be inserted through a catheter into the aneurysm sac after insertion of the net or mesh; wherein the net or mesh and the neck bridge are configured to be connected to each other in the aneurysm sac; and wherein the net or mesh is configured to be expanded by the insertion of embolic members into the net or mesh.

In an example, a method for intrasaccular occlusion of a cerebral aneurysm can comprise: inserting a guidewire into an aneurysm sac; inserting a catheter over the guidewire into the aneurysm sac; inserting a distal flexible net or mesh through the catheter into the aneurysm sac; inserting a proximal neck bridge through the catheter into the aneurysm sac between the net or mesh and the aneurysm neck, wherein the neck bridge expands in the aneurysm sac after it exits the catheter; inserting embolic members through the catheter into the net or mesh, wherein accumulation of the embolic members in the net or mesh expands the net or mesh; and withdrawing the catheter from the aneurysm sac.

In an example, a neck bridge can be made from a tubular and/or columnar mesh by radially-constricting the mesh with two annular members (e.g. rings, bands, washers, circular wires, ties, cylinders, strings, or tubes). In an example, a neck bridge can be made from a tubular mesh by: radially-constricting the mesh with an annular member (e.g. ring, band, washer, circular wire, tie, cylinder, string, or tube) at a location between 25% and 50% of the length of the mesh from the distal end of the mesh; and then everting the proximal end of the mesh over the distal end (or inverting the distal end of the mesh into the remaining proximal portion of the mesh).

In an example, an annular member can be a composite member comprising an outer member (e.g. outer ring) which goes around the outside the tubular mesh and an inner member (e.g. inner ring) which goes into the tubular mesh, wherein embolic members are inserted through the opening in the inner member. In an example, one or more annular members can be glued and/or adhered to a tubular mesh. In an example, one or more annular members can be slid and/or fastened around the outside of a tubular mesh.

In an example, a neck bridge can be made by radially-constricting, inverting, everting, folding, and/or compressing a tubular mesh. In an example, a neck bridge can have two layers which are made by inverting, everting, folding, and/or compressing a single tubular mesh. In an example, a method for making a neck bridge can comprise: radially-constricting a distal portion of a columnar or frustum-shaped mesh; and then everting the proximal portion of the mesh over the distal portion of the mesh and/or inverting the distal portion of the mesh into the proximal portion of the mesh.

In an example, a neck bridge can be made from a tubular mesh by: radially-constricting a distal portion of the mesh; then everting the proximal end of the mesh over the distal end (or inverting the distal end of the mesh into the proximal portion of the mesh); radially-constricting the currently-distal portion (which was a proximal portion before eversion); and then inverting the currently-distal portion into the currently-proximal portion.

In an example, a proximal neck bridge can be formed from a (tubular) mesh, a first annular member (e.g. ring, band, washer, circular wire, tie, cylinder, string, or tube), and second annular member (e.g. ring, band, washer, circular wire, tie, cylinder, string, or tube), wherein the first annular member is fastened around a distal portion of the mesh in order to radially-constrict the distal portion of the mesh, a proximal portion of the mesh is everted over the annular member and the distal portion of the mesh, the second annular member is fastened around the distal (previously-proximal) portion of the mesh in order to radially-constrict the distal (previously-proximal) portion of the mesh, and then the distal portion of the mesh is inverted (e.g. compressed) into the proximal portion of the mesh, thereby forming a double-layer bowl-shaped mesh.

In an example, a neck bridge can be made from a tubular mesh by: radially-constricting the mesh at a location between 25% and 50% of the length of the mesh from the distal end of the mesh; and then everting the proximal end of the mesh over the distal end (or inverting the distal end of the mesh into the remaining proximal portion of the mesh). In an example, a neck bridge can be made by 3D printing. In an example, a proximal neck bridge can be made with metal and a distal net or mesh can be made with a polymer.

In an example, a neck bridge can have a shape with a distal-facing concavity. In an example, a neck bridge can have a composite shape comprising an inner, smaller, bowl-shaped portion and an outer, larger, bowl-shaped portion, wherein the inner and outer portions are formed from a common mesh by radially-constricting, inverting, and/or everting the common mesh. In an example, a neck bridge can have a compound-bowl shape, comprising a larger outer bowl portion and an inner bowl portion, wherein the diameter of the outer bowl portion is 2-5 times the size of the inner bowl portion.

In an example, a neck bridge can have a shape with concave portions and convex portions. In an example, a neck bridge can have a six-sided or eight-sided star-shaped cross-sectional perimeter while it is being delivered through a catheter to an aneurysm sac.

In an example, a central portion of a neck bridge can be less elastic, less flexible, more resilient, and/or have a higher durometer than the rest of the neck bridge. In an example, a central portion of a neck bridge can be more porous, have larger holes, and/or have a wider weave than the rest of the neck bridge. In an example, a neck bridge can comprise a porous metal mesh layer and a non-porous polymer layer. In an example, a neck bridge can have two layers of material and/or mesh, wherein a first layer is more-flexible, more-elastic, and/or lower-durometer than a second layer.

In an example, a perimeter (e.g. circumferential) of a neck bridge can be more porous, have larger holes, and/or have a wider weave than the rest of the neck bridge. In an example, a peripheral (e.g. non-central) portion of a neck bridge can be more porous, have larger holes, and/or have a wider weave than a central portion of the neck bridge. In an example, a proximal neck bridge and a distal net or mesh can be made from a single piece of material (e.g. a single piece of mesh) with non-uniform porosity, elasticity, thickness, and/or weave.

In an example, a proximal portion (e.g. the proximal half or third) of a net or mesh can be more porous, have larger holes, and/or have a wider weave than the rest of the net or mesh. In an example, the central third of a neck bridge can be less porous, have smaller holes, and/or have a denser weave than the rest of the neck bridge. In an example, the thickness of a proximal portion of a woven neck bridge can be greater than that of a distal portion of the neck bridge.

In an example, a single-layer neck bridge can be made by 3D printing a hemispherical, hemi-ellipsoidal, and/or bowl-shaped resilient mesh. In an example, a neck bridge can have two layers of material and/or mesh, wherein the distance between the layers is greater in the center of the neck bridge than in the periphery of the neck bridge. In an example, a neck bridge can have two substantially-parallel layers of material and/or mesh.

In an example, a net or mesh can be made by weaving or braiding metal tubes or wires. In an example, a distal net or mesh can be made by weaving and/or braiding a combination of metal and polymer filaments (e.g. wires, strands, tubes, yarns, and/or threads). In an example, a distal net or mesh can be made with a lower percentage of metal (components) than a neck bridge. In an example, a net or mesh can be made by 3D printing with an elastomeric polymer material. In an example, a net or mesh can be made by weaving or braiding polymer tubes, strands, strips, or yarns, or threads.

In an example, a distal net or mesh can be made by radially-constricting the ends of a tubular mesh. In an example, a distal net or mesh can be made by radially-constricting proximal and distal portions (e.g. proximal and distal ends) of a tubular mesh, wherein this forms a globular net or mesh. In an example, a distal net or mesh can be made by radially-constricting end portions of a tubular mesh with rings, bands, circular wires, strings, or ties.

In an example, a distal net or mesh can be made by radially-constricting and inverting proximal and distal portions (e.g. proximal and distal ends) of a tubular mesh, wherein this forms a globular net or mesh. In an example, a distal net or mesh can be made by: radially-constricting and inverting the proximal end of a tubular mesh; and radially-constricting and everting the distal end of the tubular mesh, such that both ends face in a distal direction.

In an example, a distal net or mesh can be convex before expansion and can have both concave and convex portions after expansion. In an example, the surface of a distal net or mesh can include both concave and convex portions. In an example, a distal net or mesh can have a star-shaped cross-sectional perimeter while it is being delivered through a catheter to an aneurysm sac.

In an example, a distal net or mesh can be less resilient, more flexible, more compliant, and/or made with lower-durometer material than a neck bridge. In an example, a distal net or mesh can be sufficiently compliant, elastic, flexible, and/or low-durometer so that it can conform to the shape of even a multi-lobed aneurysm sac when embolic members are inserted into the net or mesh.

In an example, a distal portion (e.g. the distal half or third) of a net or mesh can be more porous, have larger holes, and/or have a wider weave than the rest of the net or mesh. In an example, a proximal portion (e.g. the proximal half or third) of a net or mesh can be thicker (e.g. be made with thicker wires, tubes, or strands or have more layers) than the rest of the net or mesh.

In an example, a distal net or mesh can be a honeycomb mesh with hexagonal holes (e.g. holes, openings, or pores). In an example, a distal net or mesh can have hexagonal holes (e.g. holes, openings, or pores) like a honeycomb.

In an example, a neck bridge can be outside a net or mesh. In an example, a neck bridge can have a shape comprising two nested bowls: a larger outer bowl portion; and a smaller inner bowl portion. In an example, a proximal neck bridge and a distal net or mesh can be (made from different pieces of material and) connected together in a nested configuration, wherein the net or mesh is (at least partially) within the concavity of the neck bridge, before they are inserted into a catheter for delivery to an aneurysm.

In an example, the proximal third of a distal net or mesh (before expansion) can be nested inside a neck bridge. In an example, a proximal portion (e.g. between 25% and 60%) of a distal net or mesh can be nested within the concavity of a proximal neck bridge. In an example, between 10% and 30% the length of a distal net or mesh can be nested within the concavity of a proximal neck bridge before expansion of the net or mesh by insertion of embolic members.

In an example, a proximal neck bridge and a distal net or mesh can be simultaneously delivered to an aneurysm sac through a catheter. In an example, a proximal neck bridge and a distal net or mesh can be attached to each other before insertion into a catheter for delivery to an aneurysm sac. In an example, a proximal neck bridge and a distal net or mesh can be made separately and attached to each other before insertion into a catheter for delivery to an aneurysm sac.

In an example, a method for providing an intrasaccular aneurysm occlusion device can comprise: forming a proximal neck bridge; forming a distal net or mesh; attaching the distal net or mesh to the proximal neck bridge to form a combined device, wherein the net or mesh is at least partially nested within a concavity of the neck bridge; and then providing the combined device to a clinician for deployment through a catheter to an aneurysm sac.

In an example, a method for providing an intrasaccular aneurysm occlusion device can comprise: forming a proximal neck bridge by radially-constricting, inverting, and/or everting a tubular mesh; forming a distal net or mesh by radially-constricting, inverting, and/or everting a tubular mesh; attaching the distal net or mesh to the proximal neck bridge to form a combined device, wherein the net or mesh is at least partially nested within a concavity of the neck bridge; and then providing the combined device to a clinician for deployment through a catheter to an aneurysm sac. In an example, a proximal neck bridge and a distal net or mesh can be (made from different pieces of material and) connected together in a nested configuration before they are inserted into a catheter for delivery to an aneurysm.

In an example, a neck bridge can have a radially-compressed configuration when in a catheter being delivered to an aneurysm sac and a radially-expanded configuration when released from the catheter into the aneurysm sac. In an example, a neck bridge can self-expand within an aneurysm sac after it exits a catheter. In an example, the size (e.g. diameter) of a neck bridge can be adjusted remotely by the application of electrical energy to the neck bridge.

In an example, a distal net or mesh can have a radially-compressed configuration when in a catheter being delivered to an aneurysm sac and a radially-expanded configuration when filled with embolic members. In an example, a net or mesh can have a folded configuration when in a catheter being delivered to an aneurysm sac. In an example, a proximal neck bridge and a distal net or mesh can be made from a single piece of material and/or can be connected together (e.g. in a nested configuration) before they are inserted into a catheter for delivery to an aneurysm.

In an example, there can be a hole (e.g. hole, opening, tube, or channel) in the center of a proximal neck bridge through which embolic members are inserted into a distal net or mesh. In an example, there can be a hole (e.g. hole, opening, tube, or channel) in the center of a neck bridge through a catheter is inserted, wherein embolic members are delivered through the catheter into a distal net or mesh. In an example, there can be a hole (e.g. hole, opening, tube, or channel) in the center of a distal net or mesh through which embolic members are inserted, thereby expanding the net or mesh.

In an example, a hole (e.g. hole, opening, or port) in the proximal surface of a neck bridge can be aligned with a hole in the proximal surface of a net or mesh so that embolic members can be inserted through the neck bridge into the net or mesh. In an example, the size of a hole on the proximal surface of a neck bridge can be between 5% and 25% of the size of the whole proximal surface of the neck bridge. In an example, the location of the hole on the proximal surface of a net or mesh can be adjusted. In an example, the person deploying the device can remotely adjust the location of a hole from a central location on the proximal surface of the neck bridge to a non-central location, or vice versa.

In an example, a proximal neck bridge and a distal net or mesh can be connected to the same wire. In an example, embolic members can be advanced into a distal net or mesh by a wire. In an example, an annular member can be detachably-connected to a catheter.

In an example, a distal net or mesh and a proximal neck bridge can be delivered separately to an aneurysm sac, wherein the proximal neck bridge is inserted into the aneurysm sac after the distal net or mesh is inserted into the aneurysm sac. In an example, a distal net or mesh and a proximal neck bridge can be delivered separately to an aneurysm sac, wherein the net or mesh is inserted through a first catheter into the aneurysm sac and then the neck bridge is inserted through a second catheter. In an example, a proximal neck bridge and a distal net or mesh can be delivered sequentially through one or more catheters to an aneurysm sac, wherein the neck bridge is delivered into the sack before the net or mesh.

In an example, a proximal neck bridge and a distal net or mesh can be delivered sequentially through one or more catheters to an aneurysm sac, wherein the net or mesh is delivered into the sack before the neck bridge. In an example, a proximal neck bridge and a distal net or mesh can be made separately, delivered separately to an aneurysm sac through one or more catheters, and then attached to each other in the aneurysm sac. In an example, a proximal neck bridge and a distal net or mesh can be made separately from different pieces of material, delivered separately through one or more catheters into an aneurysm sac, and then connected together within the aneurysm sac.

In an example, a proximal neck bridge and a distal net or mesh can be delivered separately (e.g. sequentially) through the same catheter to an aneurysm sac. In an example, a proximal neck bridge and a distal net or mesh can be delivered separately (e.g. sequentially) through the same catheter to an aneurysm sac. In an example, a proximal neck bridge and a distal net or mesh can be delivered to an aneurysm sac through one or more catheters at different times.

In an example, a method for occluding an aneurysm can comprise: inserting a distal flexible net or mesh through a catheter into an aneurysm sac; inserting a proximal neck bridge through a catheter into the aneurysm sac; connecting the net or mesh and the neck bridge together in the aneurysm sac; and then inserting embolic members through a catheter into the net or mesh. In an example, a distal net or mesh and a proximal neck bridge can be separated by a distance which is at least twice the length of the distal net or mesh while they are being delivered through a catheter to an aneurysm sac, but the net or mesh and the neck bridge are drawn together (and connected) after they are inserted into the aneurysm sac.

In an example, a distal net or mesh and a proximal neck bridge can be separate and not in a nested configuration before insertion into a catheter for delivery to an aneurysm sac. In an example, a distal net or mesh and a proximal neck bridge are not in a nested configuration (e.g. they do not overlap) while they are being delivered through a catheter to an aneurysm sac, but are moved into a nested configuration after they have been inserted in the aneurysm sac.

In an example, a distal net or mesh and a proximal neck bridge are not in a nested configuration (e.g. do not overlap) while they are being delivered through a catheter to an aneurysm sac, but are moved into a nested configuration by application of electrical energy after they have been inserted in the aneurysm sac. In an example, a proximal neck bridge and a distal net or mesh do not overlap (e.g. are not nested) as they are delivered through a catheter to an aneurysm, but do overlap (e.g. are nested) after they exit the catheter in the aneurysm sac. In an example, a proximal neck bridge and a distal net or mesh can be in a nested configuration, wherein between 25% and 75% of the net or mesh is within the concavity of the neck bridge, after they are connected to each other within an aneurysm sac.

In an example, a distal net or mesh and a proximal neck bridge are not in a nested configuration (e.g. they do not overlap) while they are being delivered through a catheter to an aneurysm sac, but are moved into a nested configuration by a wire which is pulled, pushed, or rotated after they have been inserted in the aneurysm sac. In an example, a distal net or mesh can be attached to a first wire, a proximal neck bridge can be attached to a second wire, and the proximal neck bridge can slide along the first wire, wherein pushing or pulling one wire relative to the other wire connects the net or mesh and the neck bridge together.

In an example, a method for occluding an aneurysm can comprise: making a distal flexible net or mesh; and making a proximal neck bridge; wherein the distal flexible net or mesh is configured to be inserted through a catheter into an aneurysm sac; wherein the proximal neck bridge is configured to be inserted through a catheter into the aneurysm sac after insertion of the net or mesh; wherein the net or mesh and the neck bridge are configured to be connected to each other in the aneurysm sac by a device operator (or robotic component) pushing, pulling, or rotating a wire connected to the net or mesh and/or the neck bridge; and wherein the net or mesh is configured to be expanded by the insertion of embolic members into the net or mesh.

In an example, a method for occluding an aneurysm can comprise: inserting a distal flexible net or mesh through a catheter into an aneurysm sac; inserting a proximal neck bridge through a catheter into the aneurysm sac; connecting the net or mesh and the neck bridge in the aneurysm sac by a device operator (or robotic component) pushing, pulling, or rotating a wire connected to the net or mesh and/or the neck bridge; and then inserting embolic members through a catheter into the net or mesh. In an example, a proximal neck bridge and a distal net or mesh can be longitudinally separated from each other by a wire (or other spacing mechanism) while they are delivered through a catheter to an aneurysm sac, but this longitudinal separation can be shortened and/or eliminated after they are inserted into the aneurysm sac by application of electrical energy to this wire (or other spacing mechanism).

In an example, a radially-constricting annular member on a proximal neck bridge can be connected to a radially-constricting member on a distal net or mesh remotely by a person deploying the device, wherein this connection is made by pulling or pushing a wire. In an example, a distal net or mesh can be attached to a first wire, a proximal neck bridge can be attached to a second wire, and the proximal neck bridge can slide along the first wire, wherein pushing, pulling, or rotating one wire relative to the other wire decreases the distance between the net or mesh and the neck bridge.

In an example, a method for occluding an aneurysm can comprise: inserting a distal flexible net or mesh through a catheter into an aneurysm sac; inserting a proximal neck bridge through a catheter into the aneurysm sac; drawing the net or mesh and the neck bridge together in the aneurysm sac by applying electrical energy; and then inserting embolic members through a catheter into the net or mesh. In an example, a neck bridge and a net or mesh can be connected together in an aneurysm sac by magnetic attraction.

In an example, a net or mesh can include a first portion of a connection mechanism and a neck bridge can include a second portion of the connection mechanism, wherein the first portion of the connection mechanism and the second portion of the connection mechanism fit, snap, clip, latch, stick, or interlock together, thereby connecting the net or mesh and the neck bridge to each other.

In an example, an intrasaccular device or system for occluding a cerebral aneurysm can comprise: a distal flexible net or mesh, wherein the net or mesh is inserted into an aneurysm sac, wherein the net or mesh is expanded in the aneurysm sac by insertion of embolic members into the net or mesh, and wherein there is a first portion of a connection mechanism on the net or mesh; and a proximal neck bridge, wherein the neck bridge is inserted into the aneurysm sac, wherein the neck bridge is between the net or mesh and the aneurysm neck, wherein the neck bridge expands within the aneurysm sac to span the aneurysm neck, wherein the neck bridge has a proximal hole or opening through which embolic members are inserted into the net or mesh, wherein there is a second portion of the connection mechanism on the neck bridge, and wherein the first portion of the connection mechanism and the second portion of the connection mechanism fit, snap, clip, latch, plug, stick, adhere, or interlock together, thereby connecting the net or mesh and the neck bridge to each other.

In an example, an annular member can have a first portion of a connection mechanism and a catheter can have a second portion of the connection mechanism, wherein the catheter can be reversibly-attached to the annular member for delivering embolic members through the annual member. In an example, an annular member which radially-constricts a proximal neck bridge can be reversibly-connected to an annular member which radially-constricts a distal net or mesh.

In an example, embolic members can comprise one or more string-of-pearls longitudinal strands of embolic particles, wherein a longitudinal strand of embolic particles in turn comprises embolic pieces (e.g. beads, balls, micro-sponges, pieces of foam, or hydrogels) which are connected by one or more flexible wires, coils, strings, threads, lines, or sutures). In an example, embolic members can comprise one or more string-of-pearls longitudinal strands of embolic particles, wherein a longitudinal strand of embolic particles in turn comprises embolic pieces (e.g. beads, balls, micro-sponges, pieces of foam, or hydrogels) which are connected by one or more flexible wires, coils, strings, threads, lines, or sutures), and wherein there is variation in the distance between embolic pieces along the length of a strand.

In an example, embolic members can comprise one or more string-of-pearls longitudinal strands of embolic particles, wherein a longitudinal strand of embolic particles in turn comprises embolic pieces (e.g. beads, balls, micro-sponges, pieces of foam, or hydrogels) which are connected by one or more flexible wires, coils, strings, threads, lines, or sutures), and wherein the durometer of embolic pieces decreases with distance from the end of a strand.

In an example, embolic members can be advanced into a distal net or mesh by a rotating helical member (e.g. Archimedes screw). In an example, embolic members can be embolic beads or balls. In an example, embolic members can be pumped into a distal net or mesh by a flow of liquid (e.g. saline solution).

In an example, a distal net or mesh can have a remotely-operated closure mechanism which closes a hole (e.g. hole, opening, or channel) through the distal net or mesh after embolic members have been inserted into a distal net or mesh. In an example, a closure mechanism can be a bi-leaflet valve. In an example, a closure mechanism can be a movable plug. In an example, a closure mechanism can be a spring. In an example, a closure mechanism can be activated automatically when a catheter is withdrawn from the neck bridge.

In an example, a closure mechanism can be an elastic ring or band. In an example, an intrasaccular device or system for occluding a cerebral aneurysm can comprise: a proximal neck bridge which expands within an aneurysm sac and spans the aneurysm neck; a distal flexible net or mesh which is expanded within the aneurysm sac between the proximal neck bridge and the aneurysm dome; and longitudinal strands of connected embolic pieces (e.g. beads, balls, micro-sponges, pieces of foam, and/or hydrogels) which are inserted into the net or mesh, wherein insertion of the embolic members expands the net or mesh.

In an example, an intrasaccular device or system for occluding a cerebral aneurysm can comprise: a proximal neck bridge which expands within an aneurysm sac and spans the aneurysm neck; a distal flexible net or mesh which is expanded within the aneurysm sac between the proximal neck bridge and the aneurysm dome; and embolic members which are inserted into the net or mesh, wherein insertion of the embolic members expands the net or mesh.

In an example, a method for intrasaccular occlusion of a cerebral aneurysm can comprise: inserting a distal flexible net or mesh and a proximal neck bridge into an aneurysm sac, wherein the net or mesh is distal (e.g. closer to the aneurysm dome) relative to the neck bridge, and wherein the neck bridge expands in the aneurysm sac; and inserting embolic members into the net or mesh, wherein accumulation of the embolic members in the net or mesh expands the net or mesh.

In an example, a method for occluding an aneurysm can comprise: making a distal flexible net or mesh; and making a proximal neck bridge; wherein the distal flexible net or mesh is configured to be inserted through a catheter into an aneurysm sac; wherein the proximal neck bridge is configured to be inserted through a catheter into the aneurysm sac after insertion of the net or mesh; wherein the net or mesh and the neck bridge are configured to be connected to each other in the aneurysm sac by applying electrical energy; and wherein the net or mesh is configured to be expanded by the insertion of embolic members into the net or mesh.

In an example, a method for intrasaccular occlusion of a cerebral aneurysm can comprise: inserting a guidewire into an aneurysm sac; inserting a catheter over the guidewire into the aneurysm sac; inserting a distal flexible net or mesh and a proximal neck bridge through the catheter into the aneurysm, wherein the net or mesh is distal (e.g. closer to the aneurysm dome) relative to the neck bridge, and wherein the neck bridge expands in the aneurysm sac after it exits the catheter; inserting embolic members through the catheter into the net or mesh, wherein accumulation of the embolic members in the net or mesh expands the net or mesh; and withdrawing the catheter from the net or mesh and the neck bridge.

In an example, a proximal neck bridge can be part of an intrasaccular system or device for occluding a cerebral aneurysm. In an example, a neck bridge can be made by weaving and/or braiding wires, tubes, and/or filaments. In an example, a neck bridge can be made from shape-memory material.

In an example, a neck bridge can be made from a tubular mesh by: radially-constricting a distal portion of the mesh with an annular member (e.g. ring, band, washer, circular wire, tie, cylinder, string, or tube); and then everting the proximal end of the mesh over the distal end (or inverting the distal end of the mesh into the proximal portion of the mesh). In an example, a proximal neck bridge can be formed from a (tubular) mesh and an annular member (e.g. a ring, band, washer, circular wire, tie, cylinder, string, or tube), wherein first the annular member is fastened around the mesh in order to radially-constrict the mesh and then a proximal portion of the mesh is everted over the annular member and the distal portion of the mesh.

In an example, a neck bridge can be made from a tubular mesh by radially-constricting a distal portion of the mesh with an annular member (e.g. ring, band, washer, circular wire, tie, cylinder, string, or tube) and then everting the proximal end of the mesh over the distal end (or inverting the distal end of the mesh into the proximal portion of the mesh), wherein the annular member comprises two nested (e.g. concentric) rings, bands, cylinders, or tubes, and wherein the tubular mesh passes between the two nested rings, bands, cylinders, or tubes. In an example, one or more annular members can be held on a tubular mesh by tension and/or friction.

In an example, a neck bridge can be made by radially-constricting, inverting, everting, folding, and/or compressing a globular mesh. In an example, a neck bridge can have two layers which are made by inverting, everting, folding, and/or compressing a single globular mesh. In an example, a multi-layer neck bridge can be made by: forming a globular (e.g. spherical or ellipsoidal) resilient mesh; inverting and/or compressing a distal portion (e.g. the distal half) of the mesh into a proximal portion (e.g. the proximal half) of the mesh; and then connecting the distal and proximal halves together.

In an example, a neck bridge can be made from a tubular mesh by radially-constricting a distal portion of the mesh with an two-part annular member and then everting the proximal end of the mesh over the distal end (or inverting the distal end of the mesh into the proximal portion of the mesh); wherein the two-part annular member comprises a inner annular member (e.g. inner ring, band, washer, circular wire, tie, cylinder, string, or tube) and an outer annular member (e.g. outer ring, band, washer, circular wire, tie, cylinder, string, or tube); wherein the mesh passes between the inner annular member and the outer annular member; and wherein embolic members are inserted through the inner annular member.

In an example, a method for making a neck bridge can comprise: radially-constricting a tubular mesh at a location which is X % of the length of the mesh from the distal end of the mesh; and then everting the remaining (100%−X %) proximal portion of the mesh over the distal portion of the mesh and/or inverting the distal portion of the mesh into the remaining proximal portion of the mesh, wherein X % is between 5% and 40%. In an example, a multi-layer neck bridge can be made by: 3D printing a globular (e.g. spherical or ellipsoidal) resilient mesh; inverting and/or compressing a distal portion (e.g. the distal half) of the mesh into a proximal portion (e.g. the proximal half) of the mesh; and then connecting the distal and proximal portions at a proximal location.

In an example, a neck bridge can have a bowl shape, a cup shape, a hemispherical shape, and/or a hemi-ellipsoidal shape. In an example, a neck bridge can have a tulip shape. In an example, a neck bridge can have a composite shape comprising an inner, smaller, bowl-shaped portion and an outer, larger, bowl-shaped portion, wherein the inner and outer portions are formed from a tubular mesh by radially-constricting, inverting, and/or everting the tubular mesh.

In an example, a neck bridge can have a compound-bowl shape, comprising a larger outer bowl portion and a smaller inner bowl portion. In an example, a neck bridge can have a shape with a convex central portion and a concave peripheral portion. In an example, a neck bridge can have a star-shaped cross-sectional perimeter while it is being delivered through a catheter to an aneurysm sac.

In an example, a central portion of a neck bridge can be thicker (e.g. be made with thicker wires, tubes, or strands or have more layers) than the rest of the neck bridge. In an example, a central portion of a neck bridge can be thinner (e.g. be made with thinner wires, tubes, or strands or have fewer layers) than the rest of the neck bridge. In an example, a neck bridge can comprise a distal metal mesh layer, a proximal metal mesh layer, and a non-porous polymer layer between the distal metal mesh layer and the proximal metal mesh layer.

In an example, a neck bridge can comprise a resilient ring (or band) around the distal perimeter (e.g. distal circumference) of the neck bridge which helps to self-expand the neck bridge and/or engage the perimeter of the aneurysm sac to hold the neck bridge in place, wherein the resilient ring is thicker, stronger, less-compliant, less-flexible, and/or higher-durometer than the rest of the neck bridge. In an example, a perimeter (e.g. circumferential) of a neck bridge can be thicker (e.g. be made with thicker wires, tubes, or strands or have more layers) than the rest of the neck bridge.

In an example, a peripheral (e.g. non-central) portion of a neck bridge can be thicker (and/or made with thicker wires, tubes, or strands) than a central portion of the neck bridge. In an example, a peripheral (e.g. non-central) portion of a net or mesh can be more porous, have larger holes, and/or have a wider weave than a central portion of the net or mesh. In an example, a proximal neck bridge can be less porous, more densely woven, and/or thicker than a distal net or mesh.

In an example, a resilient ring or band around the perimeter (e.g. circumference) of a neck bridge can be less elastic, less flexible, more resilient, and/or have a higher durometer than the rest of the neck bridge. In an example, the perimeter (e.g. circumference) of a neck bridge can be less elastic, less flexible, more resilient, and/or have a higher durometer than the rest of the neck bridge. In an example, the weave density and/or porosity of a neck bridge can be adjusted remotely by the application of electrical energy to the neck bridge.

In an example, a neck bridge can comprise a single layer of material and/or mesh. In an example, a neck bridge can comprise two layers of material and/or mesh. In an example, a neck bridge can have two layers of material and/or mesh, wherein the distance between the layers is less in the center of the neck bridge than in the periphery of the neck bridge.

In an example, a distal net or mesh can be formed by 3D printing. In an example, a distal net or mesh can be made from one or more polymers and a neck bridge can be made from one or more metals. In an example, a distal net or mesh can be made by making holes in a compliant balloon. In an example, a net or mesh can be made by 3D printing with a polymer material.

In an example, a distal net or mesh can be made by radially-constricting proximal and distal portions of a tubular mesh. In an example, a distal net or mesh can be made by radially-constricting proximal and distal ends of a tubular mesh, wherein both ends face in distal direction after being constricted. In an example, a distal net or mesh can be made by radially-constricting end portions of a frustum-shaped mesh with rings, bands, circular wires, strings, or ties.

In an example, a distal net or mesh can be made by radially-constricting and inverting one end of a tubular mesh and radially-constricting and everting the other end of the tubular mesh, wherein this forms a globular net or mesh. In an example, a distal net or mesh can be radially-constricted while being delivered through a catheter to an aneurysm sac and be radially-expanded after exiting the catheter within the aneurysm sac.

In an example, the net or mesh can have a generally ellipsoidal shape in a first configuration, a generally globular shape in a second configuration, and the shape of the aneurysm sac in a third configuration. In an example, a distal net or mesh can be generally globular (e.g. spherical or ellipsoidal) before expansion and can have both concave and convex portions after expansion. In an example, the surface of a distal net or mesh can include a plurality of concave and convex portions. In an example, a distal net or mesh can have an undulating (e.g. sinusoidal) cross-sectional perimeter while it is being delivered through a catheter to an aneurysm sac.

In an example, a distal net or mesh can be more porous, less densely woven, and/or thinner than a neck bridge. In an example, a distal portion (e.g. the distal half or third) of a net or mesh can be more elastic, more flexible, less resilient, and/or have a lower durometer than the rest of the net or mesh. In an example, a peripheral (e.g. non-central) portion of a net or mesh can be thicker (and/or made with thicker wires, tubes, or strands) than a central portion of the net or mesh. In an example, a proximal portion (e.g. the proximal half or third) of a net or mesh can be more elastic, more flexible, less resilient, and/or have a lower durometer than the rest of the net or mesh. In an example, a distal net or mesh can be made by making holes (e.g. holes, openings, or pores) with a laser in a compliant balloon. In an example, a distal net or mesh can have hexagonal holes (e.g. holes, openings, or pores).

In an example, a distal net or mesh can be nested inside a neck bridge. In an example, a neck bridge can comprise a two-layer bowl and/or two nested bowls. In an example, a neck bridge can have a shape comprising two nested bowls, wherein this shape is formed by radially-constricting a tubular mesh at two locations to form a globular shape, and then inverting the distal half of the globular shape into the proximal half of the globular shape to create a shape comprising two nested bowls.

In an example, a proximal portion of a distal net or mesh can be nested within the concavity of a proximal neck bridge. In an example, a proximal portion (e.g. between 10% and 30%) of a distal net or mesh can be nested within the concavity of a proximal neck bridge. In an example, a proximal portion (e.g. between 25% and 60%) of a distal net or mesh can be nested within the concavity of a proximal neck bridge before expansion of the net or mesh by insertion of embolic members. In an example, between 25% and 60% the length of a distal net or mesh can be nested within the concavity of a proximal neck bridge.

In an example, a method for providing an intrasaccular aneurysm occlusion device can comprise: forming a proximal neck bridge; forming a distal net or mesh; attaching the distal net or mesh to the proximal neck bridge to form a combined device; and then providing the combined device to a clinician for deployment in an aneurysm sac. In an example, a proximal neck bridge and a distal net or mesh can be made from different pieces and then connected together before they are inserted into a catheter for delivery to an aneurysm. In an example, a proximal neck bridge and a distal net or mesh can be attached to each other before insertion into a catheter for delivery to an aneurysm sac.

In an example, a method for providing an intrasaccular aneurysm occlusion device can comprise: forming a proximal neck bridge by 3D printing; forming a distal net or mesh by radially-constricting, inverting, and/or everting a tubular mesh; attaching the distal net or mesh to the proximal neck bridge to form a combined device, wherein the net or mesh is at least partially nested within a concavity of the neck bridge; and then providing the combined device to a clinician for deployment through a catheter to an aneurysm sac.

In an example, a proximal neck bridge and a distal net or mesh can be (made from different pieces of material and) connected together in a nested configuration, wherein between 25% and 75% of the net or mesh is within the concavity of the neck bridge, before they are inserted into a catheter for delivery to an aneurysm. In an example, a proximal neck bridge and a distal net or mesh can overlap while they are delivered through a catheter to an aneurysm sac.

In an example, a neck bridge can be radially-constricted while being delivered through a catheter to an aneurysm sac and radially-expanded after exiting the catheter within the aneurysm sac. In an example, a neck bridge can have a resilient ring (or band) around the distal perimeter (e.g. distal circumference) of the neck bridge which helps to self-expand the neck bridge and/or engage the perimeter of the aneurysm sac to hold the neck bridge in place. In an example, a neck bridge can have a curled and/or rolled-up configuration when in a catheter being delivered to an aneurysm sac and an uncurled and/or unrolled configuration when released from the catheter into the aneurysm sac.

In an example, a distal net or mesh can be folded, curled, and/or rolled-up while being delivered through a catheter to an aneurysm sac and be unfolded, uncurled, and/or unrolled by being filled with embolic members after being inserted into the aneurysm sac. In an example, a distal net or mesh can have a first configuration when it is in a catheter being delivered to an aneurysm sac, a second configuration when it is released from the catheter in the aneurysm sac but before embolic members are inserted into it, and a third configuration after embolic members have been inserted into it, wherein the second configuration is larger than the first configuration, and wherein the third configuration is larger than the second configuration. In an example, a proximal neck bridge and a distal net or mesh can be made from a single piece of material.

In an example, there can be a hole (e.g. hole, opening, tube, or channel) in the center of a proximal neck bridge through which embolic members are inserted into a distal net or mesh, thereby expanding the net or mesh. In an example, there can be a hole (e.g. hole, opening, tube, or channel) in the center of a net or mesh through which embolic members are inserted. In an example, there can be a hole (e.g. hole, opening, tube, or channel) in the center of a net or mesh through a catheter is inserted, wherein embolic members are delivered through the catheter.

In an example, a neck bridge can have a plurality of holes on its proximal surface, wherein one hole can be remotely selected for inserting embolic members and the other holes can be remotely closed to prevent embolic members from escaping. In an example, a hole on the proximal surface of a net or mesh can be located on a movable (e.g. rotatable) component, enabling the location of the hole to be remotely adjusted by a person operating the device. In an example, the location of the hole on the proximal surface of a neck bridge can be adjusted. In an example, the person deploying the device can remotely adjust the location of a hole from a central location on the proximal surface of the neck bridge to a non-central location, or vice versa, by applying electrical energy to the device.

In an example, a proximal neck bridge can be connected to a first wire and a distal net or mesh can connected to a second wire. In an example, an annular member can have a thread, clip, latch, and/or opening to which a catheter can be reversibly attached.

In an example, a proximal neck bridge and a distal net or mesh can be made separately. In an example, a distal net or mesh and a proximal neck bridge can be delivered separately to an aneurysm sac, wherein the net or mesh is inserted into the aneurysm sac first and the neck bridge is inserted into the aneurysm sac second. In an example, a distal net or mesh and a proximal neck bridge can be delivered separately to an aneurysm sac, be draw close to each other within the aneurysm sac, and then be connected (e.g. attached) together within the aneurysm sac.

In an example, a proximal neck bridge and a distal net or mesh can be delivered sequentially through one or more catheters to an aneurysm sac, wherein the net or mesh is delivered into the sack before the neck bridge. In an example, a proximal neck bridge and a distal net or mesh can be sequentially delivered to an aneurysm sac through one or more catheters. In an example, a proximal neck bridge and a distal net or mesh can be delivered separately to an aneurysm sac and then attached to each other in the aneurysm sac.

In an example, a proximal neck bridge and a distal net or mesh can be made separately from different pieces of material and then connected together before delivery through a catheter to an aneurysm sac. In an example, a proximal neck bridge and a distal net or mesh can be delivered separately (e.g. sequentially) through different catheters to an aneurysm sac. In an example, a proximal neck bridge and a distal net or mesh can be delivered separately (e.g. sequentially) through different catheters to an aneurysm sac.

In an example, a proximal neck bridge and a distal net or mesh can be attached to each after they are inserted into an aneurysm sac. In an example, a distal net or mesh and a proximal neck bridge can be separated by a distance which is at least the length of the distal net or mesh while they are being delivered through a catheter to an aneurysm sac, but the net or mesh and the neck bridge are drawn together (and connected) after they are inserted into the aneurysm sac.

In an example, a distal net or mesh and a proximal neck bridge can be separate and not in a nested configuration as they are deployed through a catheter into an aneurysm sac. In an example, a distal net or mesh and a proximal neck bridge are not in a nested configuration (e.g. they do not overlap) and not directly connected while they are being delivered through a catheter to an aneurysm sac, but are moved into a nested configuration and connected together after they have been inserted in the aneurysm sac.

In an example, a proximal neck bridge and a distal net or mesh do not overlap (e.g. are not nested) as they are delivered through a catheter to an aneurysm, but do overlap (e.g. are nested) after they exit the catheter in the aneurysm sac, wherein the neck bridge and net or mesh are moved closer to each other after they exit the catheter by movement of a wire, application of electrical energy, and/or movement of shape memory material once unconstricted by the catheter. In an example, a proximal neck bridge and a distal net or mesh do not overlap (e.g. are not nested) as they are delivered through a catheter to an aneurysm, but do overlap (e.g. are nested) after they exit the catheter in the aneurysm sac.

In an example, a distal net or mesh can be attached to a first wire, a proximal neck bridge can be attached to a second wire, and the proximal neck bridge can slide along the first wire, wherein pushing, pulling, or rotating one wire relative to the other wire connects the neck bridge to the net or mesh. In an example, a distal net or mesh can be attached to a first wire, a proximal neck bridge can be attached to a second wire, and the proximal neck bridge can slide along the first wire, wherein pushing or pulling one wire relative to the other wire draws two parts of a connection mechanism (e.g. snap, clip, latch, threads, plug, pin, or magnet) together, thereby connecting the net or mesh and the neck bridge together.

In an example, a method for occluding an aneurysm can comprise: making a distal flexible net or mesh; and making a proximal neck bridge; wherein the distal flexible net or mesh is configured to be inserted through a catheter into an aneurysm sac; wherein the proximal neck bridge is configured to be inserted through a catheter into the aneurysm sac after insertion of the net or mesh; wherein net or mesh and the neck bridge are configured to be drawn close together in the aneurysm sac by a device operator (or robotic component) pulling or pushing a wire connected to the net or mesh and/or the neck bridge; and wherein the net or mesh is configured to be expanded by the insertion of embolic members into the net or mesh.

In an example, a method for occluding an aneurysm can comprise: inserting a distal flexible net or mesh through a catheter into an aneurysm sac; inserting a proximal neck bridge through a catheter into the aneurysm sac; connecting the net or mesh and the neck bridge in the aneurysm sac by a device operator (or robotic component) pushing, pulling, or rotating a wire connected to the net or mesh and/or the neck bridge; and then inserting embolic members through a catheter into the net or mesh. In an example, a proximal neck bridge can be connected to a distal net or mesh remotely by a person deploying the device, wherein this connection is made by pulling or pushing a wire.

In an example, a radially-constricting annular member on a proximal neck bridge can be connected to a radially-constricting member on a distal net or mesh remotely by a person deploying the device, wherein this connection is made by rotating a wire. In an example, a distal net or mesh can be attached to a first wire, a proximal neck bridge can be attached to a second wire, and the proximal neck bridge can slide along the first wire, wherein pushing or pulling one wire relative to the other wire changes the distance between the net or mesh and the neck bridge.

In an example, a method for occluding an aneurysm can comprise: inserting a distal flexible net or mesh through a catheter into an aneurysm sac; inserting a proximal neck bridge through a catheter into the aneurysm sac; connecting the net or mesh and the neck bridge in the aneurysm sac by a device operator (or robotic component) applying electrical energy; and inserting embolic members through a catheter into the net or mesh. In an example, a neck bridge and a net or mesh can be connected together in an aneurysm sac by adhesion or melting.

In an example, a proximal neck bridge can be connected to a distal net or mesh remotely by a person deploying the device, wherein this connection is made by application of electrical energy. In an example, the proximal portions of a neck bridge and a net or mesh can be connected to each other and the neck bridge, the net or mesh, or both can be removably-attached to a catheter.

In an example, an annular member on a distal net or mesh can function as a first part of a connection mechanism and an annular member on a proximal neck bridge can function as a second part of the connection mechanism, wherein the distal net or mesh and the proximal neck bridge are connected together as the two parts of this connection mechanism are joined together within an aneurysm sac.

In an example, embolic members can comprise one or more string-of-pearls longitudinal strands of embolic particles, wherein a longitudinal strand of embolic particles in turn comprises embolic pieces (e.g. beads, balls, micro-sponges, pieces of foam, or hydrogels) which are connected by one or more flexible wires, coils, strings, threads, lines, or sutures), and wherein there is variation in the size of embolic pieces along the length of a strand.

In an example, embolic members can comprise one or more string-of-pearls longitudinal strands of embolic particles, wherein a longitudinal strand of embolic particles in turn comprises embolic pieces (e.g. beads, balls, micro-sponges, pieces of foam, or hydrogels) which are connected by one or more flexible wires, coils, strings, threads, lines, or sutures), and wherein the distance between embolic pieces increases with distance from the end of a strand. In an example, embolic pieces in a string-of-pearls longitudinal strand can be soft, compliant, compressible, and/or low durometer.

In an example, embolic members can be advanced into a distal net or mesh by a conveyor belt mechanism. In an example, embolic members can be embolic coils or tubular polymer strands. In an example, embolic members can be separate (e.g. non-connected) embolic pieces (e.g. embolic spheres, beads, balls, polyhedrons, micro-sponges, or hydrogels).

In an example, a closure mechanism can operate without the need for activation by the person doing the procedure. In an example, a neck bridge can have a closure mechanism which closes a hole (e.g. hole, opening, or channel) through the neck bridge after embolic members have been inserted into a distal net or mesh. In an example, a closure mechanism can be a bolus of adhesive and/or congealing substance. In an example, a closure mechanism can be a one-way valve. In an example, a closure mechanism can be a tri-leaflet valve. In an example, a closure mechanism can be activated remotely by a person operating the device.

In an example, a closure mechanism can be electrolytic melting. In an example, an intrasaccular device or system for occluding a cerebral aneurysm can comprise: a guidewire which is inserted into an aneurysm sac; at least one catheter which is advanced along the guidewire into the aneurysm sac; a proximal neck bridge which is delivered through the at least one catheter, expands within an aneurysm sac, and spans the aneurysm neck; a distal flexible net or mesh which is delivered through the at least one catheter and expanded within the aneurysm sac between the proximal neck bridge and the aneurysm dome; and embolic members which are inserted into the net or mesh, wherein insertion of the embolic members expands the net or mesh.

In an example, an intrasaccular system or device for occluding a cerebral aneurysm can comprise: a distal net or mesh which is inserted into an aneurysm sac; a proximal neck bridge which is inserted into the aneurysm sac between the distal net or mesh and the aneurysm neck; embolic members which are inserted into the distal net or mesh, thereby expanding the distal net or mesh; a first annular member (e.g. ring, band, washer, circular wire, tie, cylinder, string, or tube) attached to the distal net or mesh; a second annular member (e.g. ring, band, washer, circular wire, tie, cylinder, string, or tube) attached to the proximal neck bridge; a catheter through which embolic members are inserted into the distal net or mesh; and one or more wires which push the distal net or mesh and the proximal neck bridge through the catheter into the aneurysm sac.

In an example, a method for intrasaccular occlusion of a cerebral aneurysm can comprise: inserting a distal flexible net or mesh into an aneurysm sac; inserting a proximal neck bridge into the aneurysm sac between the net or mesh and the aneurysm neck, wherein the neck bridge expands in the aneurysm sac; and inserting embolic members into the net or mesh, wherein accumulation of embolic members in the net or mesh expands the net or mesh.

In an example, a method for occluding an aneurysm can comprise: making a distal flexible net or mesh; and making a proximal neck bridge; wherein the distal flexible net or mesh is configured to be inserted through a catheter into an aneurysm sac; wherein the proximal neck bridge is configured to be inserted through a catheter into the aneurysm sac after insertion of the net or mesh; wherein net or mesh and the neck bridge are configured to be drawn close together in the aneurysm sac by applying electrical energy; and wherein the net or mesh is configured to be expanded by the insertion of embolic members into the net or mesh.

In an example, a proximal neck bridge can be used in combination with a distal net or mesh to comprise an intrasaccular system or device for occluding a cerebral aneurysm. In an example, a neck bridge can be made by weaving and/or braiding a combination of metal and polymer filaments (e.g. wires, strands, tubes, yarns, and/or threads). In an example, a proximal neck bridge can be made with a higher percentage of metal (components) than a distal net or mesh.

In an example, a neck bridge can be formed by radially-constricting a tubular mesh with one or more annular members (e.g. rings, bands, washers, circular wires, ties, cylinders, strings, or tubes). In an example, a neck bridge can be made from a tubular mesh by: radially-constricting the mesh with an annular member (e.g. ring, band, washer, circular wire, tie, cylinder, string, or tube) at a location between 5% and 40% of the length of the mesh from the distal end of the mesh; and then everting the proximal end of the mesh over the distal end (or inverting the distal end of the mesh into the remaining proximal portion of the mesh).

In an example, a proximal neck bridge can be formed from a (tubular) mesh and an annular member (e.g. a ring, band, washer, circular wire, tie, cylinder, string, or tube) which is fastened around the mesh in order to radially-constrict the mesh. In an example, an annular member (e.g. ring, band, or tube) in a neck bridge can be aligned with an annular member (e.g. ring, band, or tube) in a net or mesh so that embolic members can be inserted through both annular members into the net or mesh. In an example, one or more annular members can be melted and/or soldered to a tubular mesh.

In an example, a neck bridge can be made by radially-constricting, inverting, everting, folding, and/or compressing a mesh. In an example, a neck bridge can have two layers which are made by inverting, everting, folding, and/or compressing a single mesh. In an example, a method for making a neck bridge can comprise: radially-constricting a distal portion of a tubular mesh; and then everting the proximal portion of the mesh over the distal portion of the mesh and/or inverting the distal portion of the mesh into the proximal portion of the mesh.

In an example, a multi-layer neck bridge can be made by forming a globular (e.g. spherical or ellipsoidal) resilient mesh and then inverting and/or compressing a distal portion (e.g. the distal half) of the mesh into a proximal portion (e.g. the proximal half) of the mesh. In an example, a neck bridge can be made from a tubular mesh by radially-constricting a distal portion of the mesh and then everting the proximal end of the mesh over the distal end (or inverting the distal end of the mesh into the proximal portion of the mesh).

In an example, a neck bridge can be made from a tubular mesh by: radially-constricting the mesh at a location between 5% and 40% of the length of the mesh from the distal end of the mesh; and then everting the proximal end of the mesh over the distal end (or inverting the distal end of the mesh into the remaining proximal portion of the mesh). In an example, a multi-layer neck bridge can be made by 3D printing a globular (e.g. spherical or ellipsoidal) resilient mesh and then inverting and/or compressing a distal portion (e.g. the distal half) of the mesh into a proximal portion (e.g. the proximal half) of the mesh.

In an example, a neck bridge can have a hyperbolic shape or funnel shape. In an example, a neck bridge can have an inverted umbrella shape. In an example, a neck bridge can have a compound-bowl shape, comprising a larger outer bowl portion and an inner bowl portion, wherein the diameter of the outer bowl portion is 2-5 times the diameter of the inner bowl portion. In an example, a neck bridge can have a compound-bowl shape, comprising a larger outer bowl portion and a smaller inner bowl portion, wherein the concavities of the outer and inner bowls both face in a distal direction.

In an example, a neck bridge can have a shape with a concave central portion and a convex peripheral portion. In an example, a neck bridge can have an undulating (e.g. sinusoidal) cross-sectional perimeter while it is being delivered through a catheter to an aneurysm sac.

In an example, a central portion of a neck bridge can be more elastic, more flexible, less resilient, and/or have a lower durometer than the rest of the neck bridge. In an example, a central portion of a neck bridge can be less porous, have smaller holes, and/or have a denser weave than the rest of the neck bridge. In an example, a neck bridge can comprise a distal metal mesh layer, a proximal metal mesh layer, and a non-porous polymer layer between the distal metal mesh layer and the proximal metal mesh layer, wherein the non-porous polymer layer does not cover the entire proximal portion of the neck bridge.

In an example, a neck bridge can have two layers of material and/or mesh, wherein a first layer is more porous than a second layer. In an example, a perimeter (e.g. circumferential) of a neck bridge can be more elastic, more flexible, less resilient, and/or have a lower durometer than the rest of the neck bridge. In an example, a peripheral (e.g. non-central) portion of a neck bridge can be more elastic, more flexible, less resilient, and/or have a lower durometer than a central portion of the neck bridge. In an example, a peripheral portion of a neck bridge can be more elastic, more flexible, less resilient, and/or have a lower durometer than a central portion of the neck bridge.

In an example, a proximal neck bridge can be more resilient, less flexible, less compliant, and/or made with higher-durometer material than a distal net or mesh. In an example, a woven or braided distal net or mesh can have a lower-density weave or braid than a proximal neck bridge. In an example, the porosity of a proximal portion of a woven neck bridge can be less than that of a distal portion of the neck bridge. In an example, the weave density of a proximal portion of a woven neck bridge can be greater than that of a distal portion of the neck bridge.

In an example, a neck bridge can have a central portion with one layer of material and/or mesh and a peripheral portion with two layers of material and/mesh. In an example, a neck bridge can have a central portion with two layers of material and/or mesh and a peripheral portion with one layer of material and/mesh. In an example, a neck bridge can have two layers of material and/or mesh, wherein the distance between the layers varies with distance from the center of the neck bridge.

In an example, a distal net or mesh can be made by weaving and/or braiding wires, tubes, and/or filaments. In an example, a distal net or mesh can be formed by making holes (e.g. via laser) in a compliant balloon. In an example, a distal net or mesh can be made with a higher percentage of polymer (components) than a neck bridge. In an example, a net or mesh can be made by 3D printing with PDMS. In an example, a net or mesh can be made by 3D printing.

In an example, a distal net or mesh can be formed by radially-constricting, inverting, and/or everting a (tubular) woven or braided mesh. In an example, a distal net or mesh can be made by radially-constricting proximal and distal portions of a tubular mesh, wherein both ends face in distal direction after being constricted. In an example, a distal net or mesh can be made by radially-constricting end portions of a tubular mesh. In an example, a distal net or mesh can be made by radially-constricting end portions of a columnar mesh with rings, bands, circular wires, strings, or ties.

In an example, a distal net or mesh can be made by radially-constricting and inverting one end of a tubular mesh and radially-constricting and everting the other end of the tubular mesh, wherein both ends face in a distal direction, and wherein this forms a globular net or mesh. In an example, a distal net or mesh can be radially-constricted while being delivered through a catheter to an aneurysm sac and be radially-expanded after being inserted into the aneurysm sac.

In an example, the net or mesh can have a generally ellipsoidal or globular shape in a second configuration and the shape of the aneurysm sac in a third configuration. In an example, a distal net or mesh can be generally globular (e.g. spherical or ellipsoidal) before expansion and can include a plurality of concave and convex portions after expansion. In an example, a distal net or mesh can have a six-sided or eight-sided star-shaped cross-sectional perimeter while it is being delivered through a catheter to an aneurysm sac.

In an example, a distal net or mesh can be sufficiently compliant, elastic, flexible, and/or low-durometer so that it can conform to the shape of even an irregularly-shaped aneurysm sac when embolic members are inserted into the net or mesh. In an example, a distal portion (e.g. the distal half or third) of a net or mesh can be thinner (e.g. be made with thinner wires, tubes, or strands or have fewer layers) than the rest of the net or mesh. In an example, a peripheral (e.g. non-central) portion of a net or mesh can be more elastic, more flexible, less resilient, and/or have a lower durometer than a central portion of the net or mesh. In an example, a distal net or mesh can have circular holes (e.g. holes, openings, or pores). In an example, a distal net or mesh can have quadrilateral holes (e.g. holes, openings, or pores) between woven and/braided wires, tubes, and/or filaments.

In an example, a neck bridge can be made from a tubular mesh by: radially-constricting a distal portion of the mesh; everting the proximal end of the mesh over the distal end (or inverting the distal end of the mesh into the proximal portion of the mesh); radially-constricting the currently-distal portion (which was a proximal portion before eversion) to form a single-layer globular shape; and inverting the currently-distal portion into the currently-proximal portion to form a two-layer bowl shape and/or nested-bowls shape.

In an example, a neck bridge can have a composite shape comprising an inner, smaller, bowl-shaped portion and an outer, larger, bowl-shaped portion, wherein the inner and outer bowl portions are nested (e.g. coaxial and/or concentric). In an example, a neck bridge can have a shape comprising two nested bowls, wherein this shape is formed by radially-constricting a tubular mesh at two locations to form a globular shape, and then inverting the distal half of the globular shape into the proximal half of the globular shape to create a shape comprising two nested bowls, wherein both ends of the tubular mesh face in a distal direction.

In an example, the proximal half of a distal net or mesh (before expansion) can be nested inside a neck bridge. In an example, a proximal portion (e.g. between 10% and 30%) of a distal net or mesh can be nested within the concavity of a proximal neck bridge before expansion of the net or mesh by insertion of embolic members. In an example, between 10% and 30% the length of a distal net or mesh can be nested within the concavity of a proximal neck bridge. In an example, between 25% and 60% the length of a distal net or mesh can be nested within the concavity of a proximal neck bridge before expansion of the net or mesh by insertion of embolic members.

In an example, a proximal neck bridge and a distal net or mesh can be delivered simultaneously through a catheter to an aneurysm sac. In an example, a proximal neck bridge and a distal net or mesh can be made from different pieces and then connected together along their central longitudinal axes before they are inserted into a catheter for delivery to an aneurysm. In an example, a proximal neck bridge and a distal net or mesh can be made separately and then attached to each other before insertion into a catheter for delivery to an aneurysm sac.

In an example, a distal net or mesh and a proximal neck bridge can be connected in a nested configuration (e.g. the distal net or mesh partially nested within a concavity of the proximal neck bridge) before they are inserted into catheter for delivery to an aneurysm sac. In an example, a method for providing an intrasaccular aneurysm occlusion device can comprise: forming a proximal neck bridge by 3D printing; forming a distal net or mesh by 3D printing; attaching the distal net or mesh to the proximal neck bridge to form a combined device wherein the net or mesh is at least partially nested within a concavity of the neck bridge; and then providing the combined device to a clinician for deployment through a catheter to an aneurysm sac.

In an example, a proximal neck bridge and a distal net or mesh can be in a nested configuration while they are delivered through a catheter to an aneurysm sac. In an example, a proximal neck bridge and a distal net or mesh can be in a nested configuration, wherein between 25% and 75% of the net or mesh is within the concavity of the neck bridge, while they are delivered through a catheter to an aneurysm sac.

In an example, a neck bridge can be radially-constricted while being delivered through a catheter to an aneurysm sac and can radially self-expand after exiting the catheter within the aneurysm sac. In an example, a neck bridge can self-expand after it exits a catheter. In an example, a neck bridge can have a folded configuration when in a catheter being delivered to an aneurysm sac and an unfolded configuration when released from the catheter into the aneurysm sac.

In an example, a distal net or mesh can be compressed and/or constricted while being delivered through a catheter to an aneurysm sac and be expanded by being filled with embolic members after being inserted into the aneurysm sac. In an example, a net or mesh can have a curled and/or rolled-up configuration when in a catheter being delivered to an aneurysm sac.

In an example, a distal net or mesh can have a first configuration when it is in a catheter being delivered to an aneurysm sac, a second configuration when it is released from the catheter in the aneurysm sac but before embolic members are inserted into it, and a third configuration after embolic members have been inserted into it, wherein the second configuration is wider than the first configuration, and wherein the third configuration is wider than the second configuration. In an example, a proximal neck bridge and a distal net or mesh can be made from a single piece of material (e.g. a single piece of mesh).

In an example, there can be a hole (e.g. hole, opening, tube, or channel) in the center of a neck bridge through which embolic members are inserted. In an example, there can be a hole (e.g. hole, opening, tube, or channel) in the center of a neck bridge through a catheter is inserted, wherein embolic members are delivered through the catheter. In an example, there can be a hole (e.g. hole, opening, tube, or channel) in the center of a distal net or mesh through which embolic members are inserted.

In an example, there can be a hole (e.g. hole, opening, tube, or channel) in the center of a net or mesh through a catheter is inserted, wherein embolic members are delivered through the catheter. In an example, the proximal surface of a proximal neck bridge can be connected to the proximal surface of a distal net or mesh so that: a hole in the neck bridge is aligned with a hole in the net or mesh; and embolic members can be inserted into the distal net or mesh.

In an example, a neck bridge can include a movable (e.g. rotatable) component on the proximal surface of the neck bridge, wherein these is a hole (e.g. hole, opening or pore) through the neck bridge on the component, and wherein the location of the hole can be remotely changed by moving (e.g. rotating) the component. In an example, the person deploying the device can remotely adjust the location of the hole on the proximal surface of the neck bridge to a non-central location, or vice versa, by rotating a component on the proximal surface of the neck bridge. In an example, the proximal portions of a neck bridge and a net or mesh can be connected to each other and the neck bridge, the net or mesh, or both can be removably-attached to a wire.

In an example, a proximal neck bridge and a distal net or mesh can be delivered to an aneurysm sac through the same catheter. In an example, an annular member can have a thread, clip, latch, and/or opening to which a catheter can be reversibly attached, wherein the catheter can be attached to the annular member for insertion of embolic members through the annular member and then detached for withdrawal of the catheter from a person's body after embolic members have been inserted.

In an example, a distal net or mesh and a proximal neck bridge can be delivered separately to an aneurysm sac, wherein the distal net or mesh is inserted into the aneurysm sac through a hole (e.g. hole or opening) in the proximal neck bridge after the neck bridge has been inserted into the aneurysm sac. In an example, a distal net or mesh and a proximal neck bridge can be delivered separately to an aneurysm sac, wherein the net or mesh is inserted through a catheter into the aneurysm sac first and the neck bridge is inserted through the same catheter into the aneurysm sac second.

In an example, a distal net or mesh and a proximal neck bridge can be delivered separately to an aneurysm sac and then moved close to each other and connected to each other within the aneurysm sac. In an example, a proximal neck bridge and a distal net or mesh can be delivered sequentially through one or more catheters to an aneurysm sac, wherein the neck bridge is delivered into the sack before the net or mesh. In an example, a proximal neck bridge and a distal net or mesh can be made separately, delivered separately to an aneurysm sac through one or more catheters, and then attached to each other in the aneurysm sac.

In an example, a proximal neck bridge and a distal net or mesh can be delivered separately to an aneurysm sac and then attached to each other in the aneurysm sac. In an example, a proximal neck bridge and a distal net or mesh can be delivered separately (e.g. sequentially) through one or more catheters to an aneurysm sac. In an example, a proximal neck bridge and a distal net or mesh can be delivered separately (e.g. sequentially) through one or more catheters to an aneurysm sac.

In an example, a proximal neck bridge and a distal net or mesh can be longitudinally separated from each other while they are delivered through a catheter to an aneurysm sac, but this longitudinal separation can be shortened and/or eliminated after they are inserted into the aneurysm sac.

In an example, a distal net or mesh and a proximal neck bridge are not directly connected to each other while they are being delivered through a catheter to an aneurysm sac, but are moved toward each other and are directly connected after they have been inserted in the aneurysm sac. In an example, a proximal neck bridge can be connected to a distal net or mesh remotely by a person deploying the device.

In an example, a distal net or mesh and a proximal neck bridge are not in a nested configuration (e.g. they do not overlap) while they are being delivered through a catheter to an aneurysm sac, but are moved into a nested configuration by an (electromagnetic) actuator after they have been inserted in the aneurysm sac. In an example, a distal net or mesh and a proximal neck bridge can be connected together into a nested configuration (e.g. the distal net or mesh partially nested within a concavity of the proximal neck bridge) within an aneurysm sac.

In an example, a proximal neck bridge and a distal net or mesh do not overlap (e.g. are not nested) as they are delivered through a catheter to an aneurysm, but do overlap (e.g. are nested) after they exit the catheter in the aneurysm sac, wherein the neck bridge and net or mesh are moved closer to each other after they exit the catheter by movement of a wire, application of electrical energy, and/or movement of shape memory material once unconstricted by the catheter. In an example, a proximal neck bridge and a distal net or mesh can be in a nested configuration after they are connected to each other within an aneurysm sac.

In an example, a distal net or mesh and a proximal neck bridge can be attached to the same wire while they are being delivered through a catheter to an aneurysm sac, but one or both can be detached from the wire, thereby enabling them to be drawn together and connected within the aneurysm sac. In an example, a distal net or mesh can be attached to a first wire, a proximal neck bridge can be attached to a second wire, and the first wire can slide through a hole or opening in the neck bridge, wherein pushing, pulling, or rotating one wire relative to the other wire connects the neck bridge to the net or mesh.

In an example, a method for occluding an aneurysm can comprise: making a distal flexible net or mesh; and making a proximal neck bridge; wherein the distal flexible net or mesh is configured to be inserted through a catheter into an aneurysm sac; wherein the proximal neck bridge is configured to be inserted through a catheter into the aneurysm sac after insertion of the net or mesh; wherein the net or mesh and the neck bridge are configured to be connected to each other in the aneurysm sac by pushing, pulling, or rotating a wire connected to the net or mesh and/or the neck bridge; and wherein the net or mesh is configured to be expanded by the insertion of embolic members into the net or mesh.

In an example, a method for occluding an aneurysm can comprise: inserting a distal flexible net or mesh through a catheter into an aneurysm sac; inserting a proximal neck bridge through a catheter into the aneurysm sac; drawing the net or mesh and the neck bridge together in the aneurysm sac by pulling, pushing, or rotating a wire connected to the net or mesh and/or the neck bridge; and then inserting embolic members through a catheter into the net or mesh.

In an example, a proximal neck bridge and a distal net or mesh can be longitudinally separated from each other by a wire (or other spacing mechanism) while they are delivered through a catheter to an aneurysm sac, but this longitudinal separation can be shortened and/or eliminated after they are inserted into the aneurysm sac by pulling, pushing, rotating, or removing this wire (or other spacing mechanism). In an example, a proximal neck bridge can be connected to a distal net or mesh remotely by a person deploying the device, wherein this connection is made by rotating a wire.

In an example, a distal net or mesh and a proximal neck bridge can be attached to the same wire to maintain a distance between them while they are being delivered through a catheter to an aneurysm sac, but one or both (e.g. the net or mesh or the neck bridge) can be detached from the wire, thereby enabling them to be drawn together and connected together within the aneurysm sac.

In an example, a neck bridge and a net or mesh can be connected together in an aneurysm sac by rotation of a helical-thread component. In an example, a neck bridge and a net or mesh can be connected together in an aneurysm sac by a snap, clip, pin, plug, or clamp mechanism. In an example, a proximally-central portion of a proximal neck bridge and a proximally-central portion of a distal net or mesh can be connected to each other.

In an example, a radially-constricting annular member on a proximal neck bridge can be connected to a radially-constricting member on a distal net or mesh remotely by a person deploying the device, wherein this connection is made by application of electrical energy. In an example, an annular member which radially-constricts a proximal neck bridge can be connected to an annular member which radially-constricts a distal net or mesh.

In an example, embolic members can be string-of-pearl longitudinal strands of embolic pieces (e.g. embolic spheres, beads, balls, polyhedrons, micro-sponges, or hydrogels) which are interconnected by flexible strands (e.g. wires, sutures, filaments, strings, or threads). In an example, embolic members can comprise one or more string-of-pearls longitudinal strands of embolic particles, wherein a longitudinal strand of embolic particles in turn comprises embolic pieces (e.g. beads, balls, micro-sponges, pieces of foam, or hydrogels) which are connected by one or more flexible wires, coils, strings, threads, lines, or sutures), and wherein the sizes of embolic pieces decrease with distance from the end of a strand.

In an example, embolic members can comprise one or more string-of-pearls longitudinal strands of embolic particles, wherein a longitudinal strand of embolic particles in turn comprises embolic pieces (e.g. beads, balls, micro-sponges, pieces of foam, or hydrogels) which are connected by one or more flexible wires, coils, strings, threads, lines, or sutures), and wherein there is variation in the durometer of embolic pieces along the length of a strand.

In an example, embolic pieces in a string-of-pearls longitudinal strand can be centrally connected by flexible wires, coils, strings, threads, lines, or sutures. In an example, embolic members can be coils. In an example, embolic members can be micro-sponges, pieces of foam, or hydrogels. In an example, embolic members can be soft, compliant, compressible, and/or low-durometer.

In an example, a distal net or mesh can have a closure mechanism which closes a hole (e.g. hole, opening, or channel) through the distal net or mesh so that inserted embolic members do not escape. In an example, a neck bridge can have a closure mechanism which closes a hole (e.g. hole, opening, or channel) through the neck bridge so that inserted embolic members do not escape.

In an example, a closure mechanism can be a magnet. In an example, a closure mechanism can be a rotating helical member. In an example, a closure mechanism can be activated automatically when a catheter is withdrawn from the distal net or mesh. In an example, a closure mechanism can be activated remotely by a person operating the device. In an example, a closure mechanism can operate without the need for activation by the person doing the procedure.

Having now completed an introductory section which covers some of the general concepts, components, and methods which comprise this invention, this disclosure now transitions to discussion of the specific embodiments of this invention which are shown in FIGS. 1 through 22. Where relevant, the general concepts, components, and methods just discussed can be applied to variations of the embodiments which are shown in FIGS. 1 through 22.

FIGS. 1 through 3 show three views, at three different times, of an example of how a proximal neck bridge can be formed from a (tubular) mesh. This proximal neck bridge can be part of an intrasaccular system or device for occluding a cerebral aneurysm. In an example, a proximal neck bridge can be used in combination with a distal net or mesh to comprise an intrasaccular system or device for occluding a cerebral aneurysm.

FIG. 1 shows a (tubular) mesh 101 and an annular member 102 (e.g. a ring, band, washer, cylinder, tube, or circular wire) at a first time, before the annular member has been fastened around the mesh in order to radially-constrict the mesh.

FIG. 2 shows the mesh 101 and the annular member 102 at a second time, after the annular member has been fastened around a distal portion of the mesh in order to radially-constrict the distal portion of the mesh. This forms an inverted bowl-shaped mesh.

FIG. 3 shows the mesh 101 and the annular member 102 at a third time, after a proximal portion of the mesh has been everted over the annular member and the distal portion of the mesh. This forms a bowl-shaped mesh which can serve as a proximal neck bridge which is part of an intrasaccular system or device for occluding a cerebral aneurysm. Example variations discussed elsewhere in this disclosure or priority-linked disclosures can be applied to this example where relevant.

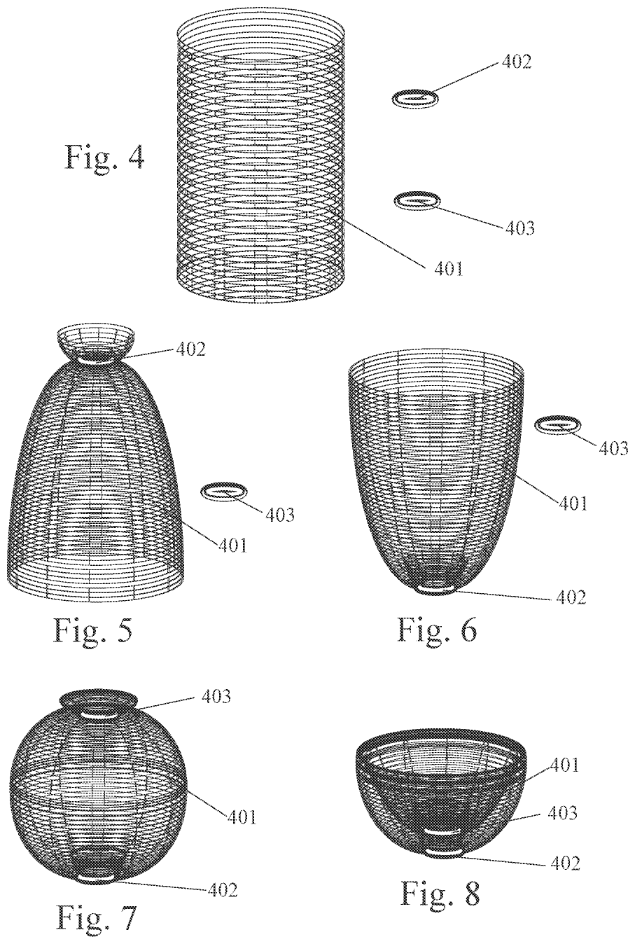

FIGS. 4 through 8 show five views, at five different times, of another example of how a proximal neck bridge can be formed from a (tubular) mesh. This proximal neck bridge can comprise part of an intrasaccular system or device for occluding a cerebral aneurysm. In an example, a proximal neck bridge can be used in combination with a distal net or mesh to form an intrasaccular system or device for occluding a cerebral aneurysm.

FIG. 4 shows a (tubular) mesh 401, first annular member 402 (e.g. ring, band, washer, cylinder, tube, or circular wire), and second annular member 403 (e.g. ring, band, washer, cylinder, tube, or circular wire) at a first time, before either annular member has been fastened around the mesh in order to radially-constrict the mesh.

FIG. 5 shows the mesh 401 and the annular members at a second time, after first annular member 402 has been fastened around a distal portion of the mesh in order to radially-constrict the distal portion of the mesh.

FIG. 6 shows the mesh 401 and the annular members at a third time, after a proximal portion of the mesh has been everted over the annular member and the distal portion of the mesh.

FIG. 7 shows the mesh 401 and the annular members at a fourth time, after second annular member 403 has been attached to the currently-distal (previously-proximal) portion of the mesh in order to radially-constrict the currently-distal (previously-proximal) portion of the mesh.

FIG. 8 shows the mesh 401 and the annular members at a fifth time, after the distal portion of the mesh has been inverted (e.g. compressed) into the proximal portion of the mesh, thereby forming a double-layer bowl-shaped mesh. This bowl-shaped mesh can serve as a proximal neck bridge as part of an intrasaccular system or device for occluding a cerebral aneurysm. Example variations discussed elsewhere in this disclosure or priority-linked disclosures can be applied to this example where relevant.

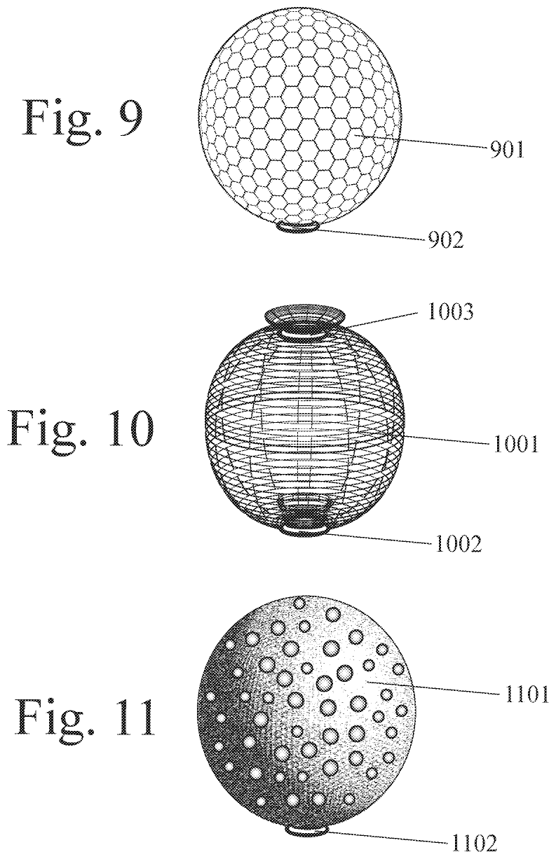

FIGS. 9 through 11 show three examples of a distal net or mesh which can be part of an intrasaccular system or device for occluding a cerebral aneurysm. In an example, a distal net or mesh can be used in combination with a proximal neck bridge to comprise an intrasaccular system or device for occluding a cerebral aneurysm.

FIG. 9 shows an example of a distal net or mesh 901 and an annular member 902 (e.g. a ring, band, washer, cylinder, tube, or circular wire) on a proximal portion of the net or mesh, wherein the net or mesh has been formed by 3D printing. In this example, the net or mesh is honeycomb mesh with hexagonal openings. Example variations discussed elsewhere in this disclosure or priority-linked disclosures can be applied to this example where relevant.

FIG. 10 shows an example of a distal net or mesh 1001 and two annular members, 1002 and 1003, (e.g. rings, bands, washers, cylinders, tubes, or circular wires) on proximal and distal portions of the net or mesh, respectively, wherein the net or mesh has been formed by radially-constricting, inverting, and/or everting a (tubular) woven or braided mesh. Example variations discussed elsewhere in this disclosure or priority-linked disclosures can be applied to this example where relevant.

FIG. 11 shows an example of a distal net or mesh 1101 and an annular member 1102 (e.g. a ring, band, washer, cylinder, tube, or circular wire) on the net or mesh, wherein the net or mesh is a compliant balloon, and wherein holes (e.g. holes or openings) have been made in the balloon. In an example, holes can be made by a laser. Example variations discussed elsewhere in this disclosure or priority-linked disclosures can be applied to this example where relevant.

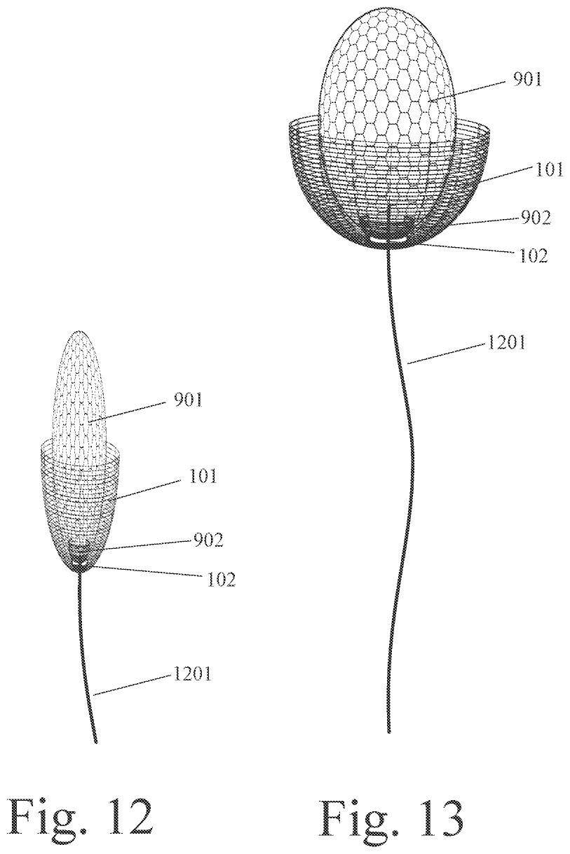

FIGS. 12 and 13 show two views, at two different times during deployment, of an example of an intrasaccular system or device for occluding a cerebral aneurysm comprising a proximal neck bridge and a distal net or mesh. In this example, the neck bridge and net or mesh are connected in a nested configuration before they are inserted into catheter for delivery to an aneurysm sac. A potential advantage of having them be connected in a nested configuration before insertion into a catheter is to avoid having to position and connect them remotely within an aneurysm sac. A potential disadvantage of having them be connected in a nested configuration before insertion into a catheter is having to push (and navigate) a greater cross-sectional mass through the catheter.

FIG. 12 shows this system or device at a first time when a proximal neck bridge 101 and a distal net or mesh 901 are in a radially-compressed configuration for insertion through a catheter (not shown) to an aneurysm sac (not shown). In this example, the system or device further comprises: a first annular member 902 (e.g. ring, band, washer, cylinder, tube, or circular wire) attached to the distal net or mesh; a second annular member 102 (e.g. ring, band, washer, cylinder, tube, or circular wire) attached to the proximal neck bridge; and a wire 1201 which pushes the combined neck bridge and net or mesh through a catheter into the aneurysm sac.

FIG. 13 shows this system or device at a second time after the proximal neck bridge 101 and the distal net or mesh 901 have exited a catheter and been inserted into an aneurysm sac (not shown), but before embolic members have been inserted into the distal net or mesh 901. At this time, the neck bridge has self-expanded within the aneurysm sac after exiting the radial constraint of the catheter. Also at this time, the distal net or mesh has partially expanded due to exiting the radial constrain of the catheter, but has not yet been fully expanded by insertion of embolic members. Example variations discussed elsewhere in this disclosure or priority-linked disclosures can be applied to this example where relevant.

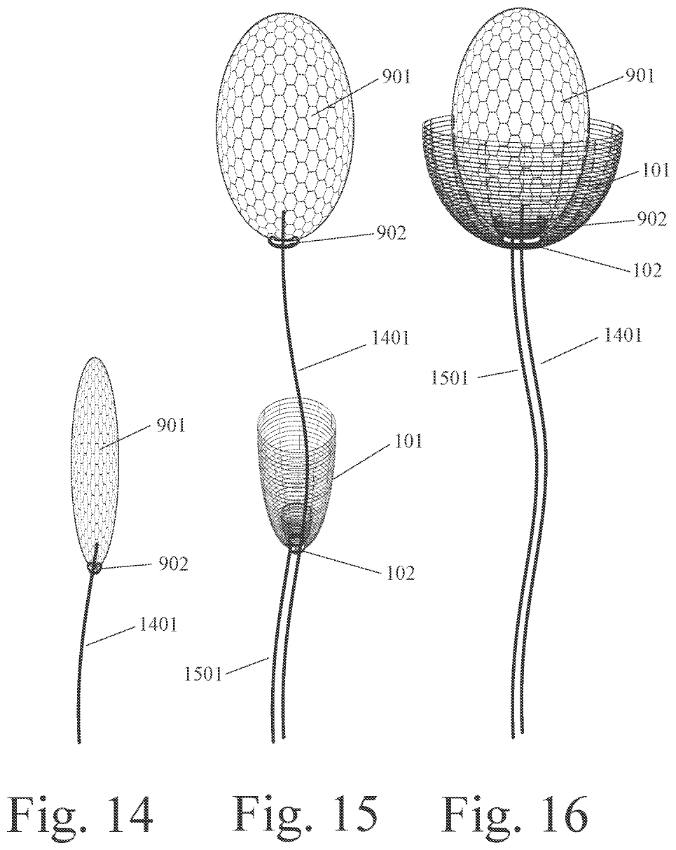

FIGS. 14 through 16 show three views, at three different times, of an example of an intrasaccular system or device for occluding a cerebral aneurysm which comprises a proximal neck bridge and a distal net or mesh. In this example, the neck bridge and the net or mesh are separate and not in a nested configuration before insertion into an aneurysm sac. A potential advantage of having them be separate and not in a nested configuration before insertion into a catheter is to decrease the cross-sectional mass which has to be pushed (and navigated) through a catheter for insertion into the aneurysm sac. A potential disadvantage of having them be separate and not in a nested configuration before insertion into a catheter is having to position and connect them remotely within the aneurysm sac.

FIG. 14 shows a distal net or mesh 901 of this system or device at a first time, when the net or mesh is in a radially-compressed configuration for insertion through a catheter (not shown). This example further comprises: a first annular member 902 (e.g. ring, band, washer, cylinder, tube, or circular wire) attached to the distal net or mesh; and a first wire 1401 which pushes the distal net or mesh through a catheter to an aneurysm sac (not shown).

FIG. 15 shows this system or device at a second time, when the distal net or mesh 901 has exited a catheter (not shown) into an aneurysm sac (not shown), but the proximal neck bridge has not. At this time, the distal net or mesh has partially expanded because it has exited a catheter and has been released from radial compression by catheter, but is not yet fully expanded by insertion of embolic members into the net or mesh. At this time, the proximal neck bridge 101 is in a compressed configuration in catheter (not shown). This system or device further comprises: a first annular member 902 (e.g. ring, band, washer, cylinder, tube, or circular wire) attached to the distal net or mesh; a second annular member 102 (e.g. ring, band, washer, cylinder, tube, or circular wire) attached to the proximal neck bridge; a first wire 1401 which pushes the net or mesh through a catheter to an aneurysm sac; and a second wire 1501 which pushes the proximal neck bridge through the catheter into the aneurysm sac.

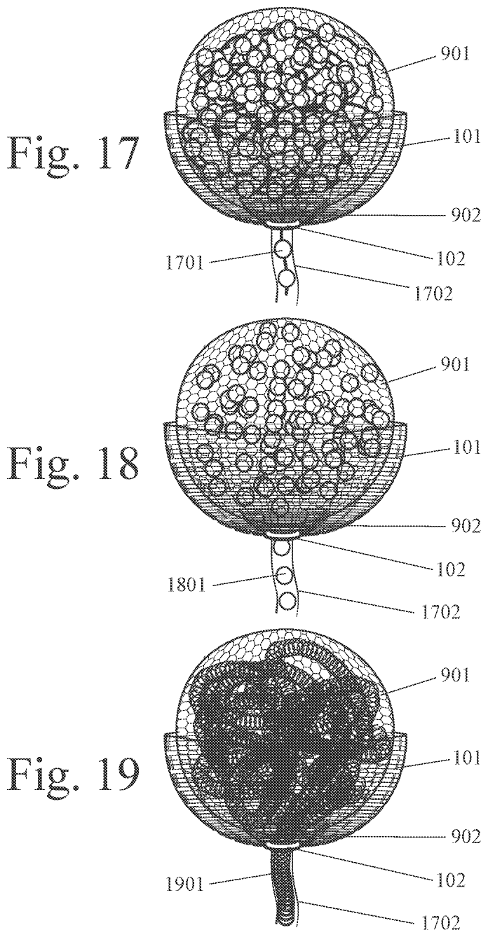

FIG. 16 shows this system or device at a third time, when the distal net or mesh 901 and the proximal neck bridge 101 have both exited a catheter (not shown) into an aneurysm sac (not shown). At this time, the distal net or mesh has partially expanded because it has exited a catheter and been released from radial compression by catheter, but is not yet fully expanded by insertion of embolic members into the net or mesh. At this time, the proximal neck bridge has self-expanded because it has exited a catheter (not shown) and been released from radial compression by the catheter. This system or device further comprises: a first annular member 902 (e.g. ring, band, washer, cylinder, tube, or circular wire) attached to the distal net or mesh; a second annular member 102 (e.g. ring, band, washer, cylinder, tube, or circular wire) attached to the proximal neck bridge; a first wire 1401 which pushes the net or mesh through a catheter to an aneurysm sac; and a second wire 1501 which pushes the proximal neck bridge through the catheter into the aneurysm sac. Example variations discussed elsewhere in this disclosure or priority-linked disclosures can be applied to this example where relevant.

FIGS. 17 through 19 show three examples of an intrasaccular system or device for occluding a cerebral aneurysm. These examples comprise: a distal net or mesh which is inserted into an aneurysm sac; a proximal neck bridge which is inserted into the aneurysm sac between the distal net or mesh and the aneurysm neck; and embolic members which are inserted into the distal net or mesh, thereby expanding the distal net or mesh. This system or device further comprises: a first annular member (e.g. ring, band, washer, cylinder, tube, or circular wire) attached to the distal net or mesh; a second annular member (e.g. ring, band, washer, cylinder, tube, or circular wire) attached to the proximal neck bridge; and a catheter through which embolic members are inserted into the distal net or mesh. In an example, this system or device can further comprise one or more wires which push the distal net or mesh and the proximal neck bridge through the catheter into the aneurysm sac.

FIG. 17 shows an example of an intrasaccular system or device for occluding a cerebral aneurysm comprising: a flexible distal net or mesh 901 which is inserted into an aneurysm sac (not shown); a proximal neck bridge 101 which is inserted into the aneurysm sac between the distal net or mesh and the aneurysm neck (not shown); and embolic members 1701 which are inserted into the distal net or mesh, thereby expanding the distal net or mesh. This example further comprises: a first annular member 902 (e.g. ring, band, washer, cylinder, tube, or circular wire) attached to the distal net or mesh; a second annular member 102 (e.g. ring, band, washer, cylinder, tube, or circular wire) attached to the proximal neck bridge; and a catheter 1702 through which embolic members are inserted into the distal net or mesh.

In this example, embolic members are “string-of-pearl” longitudinal strands of embolic pieces (e.g. embolic spheres, beads, balls, polyhedrons, micro-sponges, or hydrogels) which are interconnected by flexible strands (e.g. wires, sutures, filaments, strings, or threads). In an example, this system or device can further comprise one or more wires which push the distal net or mesh and the proximal neck bridge through the catheter into the aneurysm sac. Example variations discussed elsewhere in this disclosure or priority-linked disclosures can be applied to this example where relevant.

FIG. 18 shows an example of an intrasaccular system or device for occluding a cerebral aneurysm comprising: a flexible distal net or mesh 901 which is inserted into an aneurysm sac (not shown); a proximal neck bridge 101 which is inserted into the aneurysm sac between the distal net or mesh and the aneurysm neck (not shown); and embolic members, including 1801, which are inserted into the distal net or mesh, thereby expanding the distal net or mesh. This example further comprises: a first annular member 902 (e.g. ring, band, washer, cylinder, tube, or circular wire) attached to the distal net or mesh; a second annular member 102 (e.g. ring, band, washer, cylinder, tube, or circular wire) attached to the proximal neck bridge; and a catheter 1702 through which embolic members are inserted into the distal net or mesh.

In this example, embolic members are separate (e.g. non-connected) embolic pieces (e.g. embolic spheres, beads, balls, polyhedrons, micro-sponges, or hydrogels). In an example, this system or device can further comprise one or more wires which push the distal net or mesh and the proximal neck bridge through the catheter into the aneurysm sac. Example variations discussed elsewhere in this disclosure or priority-linked disclosures can be applied to this example where relevant.

FIG. 19 shows an example of an intrasaccular system or device for occluding a cerebral aneurysm comprising: a flexible distal net or mesh 901 which is inserted into an aneurysm sac (not shown); a proximal neck bridge 101 which is inserted into the aneurysm sac between the distal net or mesh and the aneurysm neck (not shown); and embolic members 1901 which are inserted into the distal net or mesh, thereby expanding the distal net or mesh. This example further comprises: a first annular member 902 (e.g. ring, band, washer, cylinder, tube, or circular wire) attached to the distal net or mesh; a second annular member 102 (e.g. ring, band, washer, cylinder, tube, or circular wire) attached to the proximal neck bridge; and a catheter 1702 through which embolic members are inserted into the distal net or mesh.

In this example, embolic members are embolic coils. In another example, embolic members can be tubular polymer strands. In an example, this system or device can further comprise one or more wires which push the distal net or mesh and the proximal neck bridge through the catheter into the aneurysm sac. Example variations discussed elsewhere in this disclosure or priority-linked disclosures can be applied to this example where relevant.

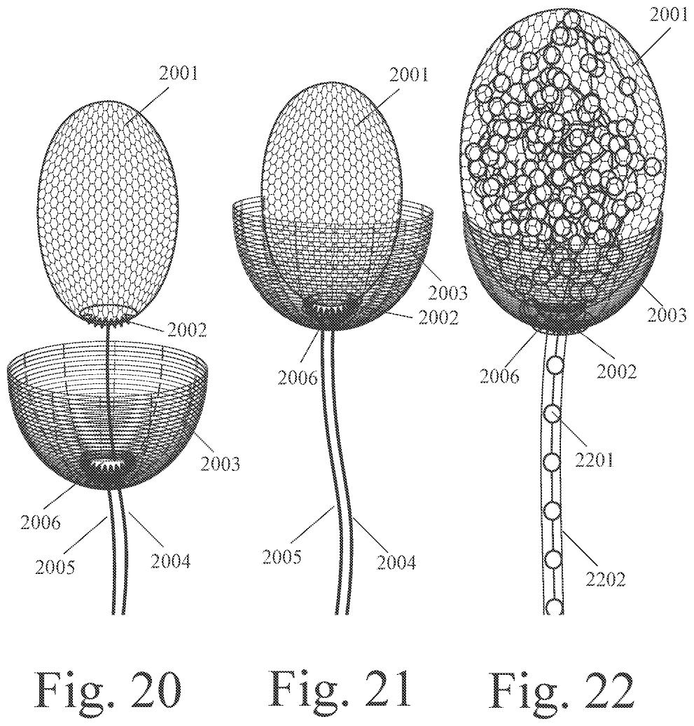

FIGS. 20 through 22 show three views, at three different times, of example of a system, device, and method for occluding a cerebral aneurysm. As a system or device, this example comprises: a flexible distal net or mesh 2001 which is inserted into an aneurysm sac (not shown); a proximal neck bridge 2003 which is inserted into the aneurysm sac between the distal net or mesh and the aneurysm neck (not shown); and embolic members 2201 which are inserted into the distal net or mesh. This example further comprises: a first annular member 2002 (e.g. ring, band, washer, cylinder, tube, or circular wire) attached to the distal net or mesh; a second annular member 2006 (e.g. ring, band, washer, cylinder, tube, or circular wire) attached to the proximal neck bridge; a catheter 2202 through which embolic members are inserted into the distal net or mesh; a first wire 2004 which pushes the distal net or mesh through a catheter into an aneurysm sac; and a second wire 2005 which pushes the proximal neck bridge through the catheter into the aneurysm sac. Example variations discussed elsewhere in this disclosure or priority-linked disclosures can be applied to this example where relevant.

In this example, the distal net or mesh and the proximal neck bridge are separate and not in a nested configuration as they are deployed through a catheter into an aneurysm sac. In this example, the distal net or mesh and the proximal neck bridge are connected together into a nested configuration within the aneurysm sac. In this example, the first annular member functions as a first part of a connection mechanism and the second annular member functions as a second part of the connection mechanism, wherein the distal net or mesh and the proximal neck bridge are connected together as the two parts of this connection mechanism are joined together within the aneurysm sac.

FIG. 20 shows this example at a first time, before the distal net or mesh and the proximal neck bridge has been connected together into a nested configuration. FIG. 21 shows this example at a second time, after the distal net or mesh and the proximal neck bridge has been connected together into a nested configuration, but before the distal net or mesh has been expanded by insertion of embolic members into the distal net or mesh. FIG. 22 shows this example at a third time, after the distal net or mesh and the proximal neck bridge have been connected together into a nested configuration and after the distal net or mesh has been expanded by insertion of embolic members into the distal net or mesh.

With respect to specific components, FIGS. 20 through 22 show a system or device for occluding a cerebral aneurysm comprising: a flexible distal net or mesh 2001 which is inserted into an aneurysm sac (not shown); a proximal neck bridge 2003 which is inserted into the aneurysm sac between the distal net or mesh and the aneurysm neck (not shown); and embolic members 2201 which are inserted into the distal net or mesh. This system or device further comprises: a first annular member 2002 (e.g. ring, band, washer, cylinder, tube, or circular wire) attached to the distal net or mesh; a second annular member 2006 (e.g. ring, band, washer, cylinder, tube, or circular wire) attached to the proximal neck bridge; a catheter 2202 through which embolic members are inserted into the distal net or mesh; a first wire 2004 which pushes the distal net or mesh through a catheter into an aneurysm sac; and a second wire 2005 which pushes the proximal neck bridge through the catheter into the aneurysm sac. Example variations discussed elsewhere in this disclosure or priority-linked disclosures can be applied to this example where relevant.

In an example, an intrasaccular device or system for occluding a cerebral aneurysm can comprise: a guidewire which is inserted into an aneurysm sac; at least one catheter which is advanced along the guidewire into the aneurysm sac; a proximal neck bridge which is delivered through the at least one catheter, expands within an aneurysm sac, and spans the aneurysm neck; a distal flexible net or mesh which is delivered through the at least one catheter and expanded within the aneurysm sac between the proximal neck bridge and the aneurysm dome; and embolic members which are inserted into the net or mesh, wherein insertion of the embolic members expands the net or mesh.

In an example, an intrasaccular device or system for occluding a cerebral aneurysm can comprise: a distal flexible net or mesh, wherein the net or mesh is inserted into an aneurysm sac, wherein the net or mesh is expanded in the aneurysm sac by insertion of embolic members into the net or mesh, and wherein there is a first portion of a connection mechanism on the net or mesh; and a proximal neck bridge, wherein the neck bridge is inserted into the aneurysm sac, wherein the neck bridge is between the net or mesh and the aneurysm neck, wherein the neck bridge expands within the aneurysm sac to span the aneurysm neck, wherein the neck bridge has a proximal hole or opening through which embolic members are inserted into the net or mesh, wherein there is a second portion of the connection mechanism on the neck bridge, and wherein the first portion of the connection mechanism and the second portion of the connection mechanism fit, snap, clip, latch, plug, stick, adhere, or interlock together, thereby connecting the net or mesh and the neck bridge to each other.

In an example, a method for intrasaccular occlusion of a cerebral aneurysm can comprise: inserting a guidewire into an aneurysm sac; inserting a catheter into the aneurysm sac; inserting a distal flexible net or mesh through the catheter into the aneurysm sac; inserting a proximal neck bridge through the catheter into the aneurysm sac between the net or mesh and the aneurysm neck, wherein the neck bridge expands in the aneurysm sac after it exits the catheter; inserting embolic members through the catheter into the net or mesh, wherein accumulation of the embolic members in the net or mesh expands the net or mesh; and withdrawing the catheter from the net or mesh and the neck bridge.

I claim:

1. An intrasaccular device or system for occluding a cerebral aneurysm comprising:
a guidewire which is inserted into an aneurysm sac;
at least one catheter which is advanced along the guidewire into the aneurysm sac;
a proximal neck bridge which is delivered through the at least one catheter, expands within an aneurysm sac, and spans the aneurysm neck;
a distal net or mesh which is delivered through the at least one catheter and expanded within the aneurysm sac between the proximal neck bridge and the aneurysm dome; and
embolic members which are inserted into the net or mesh, wherein insertion of the embolic members expands the net or mesh.

2. An intrasaccular device or system for occluding a cerebral aneurysm comprising:
a distal flexible net or mesh, wherein the net or mesh is inserted into an aneurysm sac, wherein the net or mesh is expanded in the aneurysm sac by insertion of embolic members into the net or mesh, and wherein there is a first portion of a connection mechanism on the net or mesh; and
a proximal neck bridge, wherein the neck bridge is inserted into the aneurysm sac, wherein the neck bridge is between the net or mesh and the aneurysm neck, wherein the neck bridge expands within the aneurysm sac to span the aneurysm neck, wherein the neck bridge has a proximal hole or opening through which embolic members are inserted into the net or mesh, wherein there is a second portion of the connection mechanism on the neck bridge, and wherein the first portion of the connection mechanism and the second portion of the connection mechanism fit, snap, clip, latch, plug, stick, adhere, or interlock together, thereby connecting the net or mesh and the neck bridge to each other.

3. A method for intrasaccular occlusion of a cerebral aneurysm comprising:
inserting a guidewire into an aneurysm sac;
inserting a catheter into the aneurysm sac;
inserting a distal flexible net or mesh through the catheter into the aneurysm sac;
inserting a proximal neck bridge through the catheter into the aneurysm sac between the net or mesh and the aneurysm neck, wherein the neck bridge expands in the aneurysm sac after it exits the catheter;
inserting embolic members through the catheter into the net or mesh, wherein accumulation of the embolic members in the net or mesh expands the net or mesh; and withdrawing the catheter from the net or mesh and the neck bridge.
